(19) United States
(12) Patent Application Publication
(10) Pub. No.: US 2021/0353381 A1
(43) Pub. Date: Nov. 18, 2021

(54) MEDICAL ARM SYSTEM, CONTROL DEVICE, CONTROL METHOD, AND PROGRAM

(71) Applicant: SONY GROUP CORPORATION, Tokyo (JP)

(72) Inventors: Masaru USUI, Tokyo (JP); Yohei KURODA, Tokyo (JP); Daisuke NAGAO, Tokyo (JP)

(73) Assignee: Sony Group Corporation, Tokyo (JP)

(21) Appl. No.: 17/284,795

(22) PCT Filed: Jan. 22, 2020
(86) PCT No.: PCT/JP2020/002181

§ 371 Date: Apr. 13, 2021

(30) Foreign Application Priority Data
Jan. 23, 2019 (JP) 2019-009479

(51) Int. Cl. A61B 90/50; A61B 34/00; A61B 34/35; A61B 34/20

(52) U.S. Cl. CPC A61B 90/50; A61B 34/76; A61B 2034/301; A61B 34/20; A61B 2034/2065; A61B 34/35

(57) ABSTRACT

A control device includes a control unit adapted to control an articulated medical arm configured to hold a medical instrument, where the medical instrument includes a predetermined point thereon, the control unit being adapted to control the articulated medical arm in response to a spatial relationship between the predetermined point of the medical instrument and a virtual boundary set in real space and including a target opening.

### CROSS REFERENCE TO RELATED APPLICATIONS

This application claims the benefit of Japanese Priority Patent Application JP 2019-009479 filed on Jan. 23, 2019, the entire contents of which are incorporated herein by reference.

### TECHNICAL FIELD

The present disclosure relates to a medical arm system, a control device, a control method, and a program.

### BACKGROUND ART

In recent years, in the medical field, methods of performing various operations such as surgery while observing an image of an operation site captured by an imaging device, using a balance-type arm (hereinafter referred to as “support arm”) having the imaging device held in a distal end of an arm, have been proposed. By using the balance-type arm, an affected part can be stably observed from a desired direction, and the operation can be efficiently performed.

Furthermore, technologies for setting a virtual boundary called virtual barrier or virtual wall in a real space and determining a contact between the virtual boundary with a tool held at a distal end of an arm, thereby suppressing an operation to allow the tool to enter a region beyond the barrier have been studied. For example, PTL 1 discloses an example of a technology for causing a target portion such as a medical instrument held at a distal end of an arm not to go out of a set movable region by setting a virtual wall.

### CITATION LIST

### Patent Literature

PTL 1: WO 2018/159328

### SUMMARY

### Technical Problem

Meanwhile, a virtual wall technology in related art aims at suppressing occurrence of a situation where a tool held at a distal end of an arm enters a specific region, as described above. A situation where an instrument is inserted into a body from outside the body, such as an operation of inserting an endoscope into an insertion port formed by installation of a trocar may be assumed. Therefore, there is a demand for realizing a technology for enabling improvement of the operability of an arm assuming insertion of a tool as exemplified above, as well as simply suppressing entry of the tool into a predetermined region.

Therefore, the present disclosure proposes a technology for enabling achievement of both the suppression of an operation regarding entry into a predetermined region and the improvement of the operability of an arm regarding movement to a predetermined position in a favorable manner.

### Solution to Problem

According to the present disclosure, provided is a control device, including a control unit adapted to control an articulated medical arm configured to hold a medical instrument, where the medical instrument includes (e.g. comprises) a predetermined point thereon; the control unit being adapted to control the articulated medical arm in response to a spatial relationship between the predetermined point of the medical instrument and a virtual boundary set in real space and including a target opening.

A particular example would be a control device including: a control unit configured to control an operation of a multilink structure according to a relative positional relationship between a point of action set using at least a part of the multilink structure as a reference and a virtual boundary set in a real space and having a movement target in part, the multilink structure having a plurality of links connected with each other by a joint unit, and configured to be able to hold a medical instrument.

Furthermore, according to an embodiment of the present disclosure, provided is a control method for an articulated medical arm system configured to hold a medical instrument, where the medical instrument includes a predetermined point thereon, the method including: controlling the articulated medical arm in response to a spatial relationship between the predetermined point of the medical instrument and a virtual boundary set in real space and including a target opening. A particular example would be a control method including: by a computer, controlling an operation of a multilink structure according to a relative positional relationship between a point of action set using at least a part of the multilink structure as a reference and a virtual boundary set in a real space and having a movement target in part, the multilink structure having a plurality of links connected with each other by a joint unit.

Furthermore, according to an embodiment of the present disclosure, provided is a program for causing a computer to execute the above control method. A particular example would be executing a method of controlling an operation of a multilink structure according to a relative positional relationship between a point of action set using at least a part of the multilink structure as a reference and a virtual boundary set in a real space and having a movement target in part, the multilink structure having a plurality of links connected with each other by a joint unit, and configured to be able to hold a medical instrument.

Furthermore, according to an embodiment of the present disclosure, provided is a medical arm system including an articulated medical arm configured to hold a medical instrument; and a control device as described herein.

A particular example would be a medical arm system including: a multilink structure having a plurality of links connected with each other by a joint unit, and configured to be able to hold a medical instrument; and a control unit configured to control an operation of the multilink structure according to a relative positional relationship between a point of action set using at least a part of the multilink structure as a reference and a virtual boundary set in a real space and having a movement target in part. A further particular example would be a medical arm system including: a multilink structure having a plurality of links connected with each other by a joint unit, and configured to be able to hold a medical instrument; and a control unit configured to set a virtual boundary for assisting movement of the medical instrument and control an operation of the multilink structure. A further particular example would be a medical arm system including: a multilink structure having a plurality of links connected with each other by a joint unit, and configured to be able to hold a medical instrument; and a control unit configured to control an operation of the multilink structure, in which the control unit has a first mode for assisting introduction of the medical instrument through an insertion port, and a second mode for suppressing entry of the medical instrument into a region set in a real space.

### BRIEF DESCRIPTION OF DRAWINGS

FIG. 1 is an explanatory diagram for describing an example of a schematic configuration of a medical arm device according to an embodiment of the present disclosure.

FIG. 2 is a schematic view illustrating an appearance of the medical arm device according to the embodiment.

FIG. 3 is an explanatory diagram for describing ideal joint control according to the embodiment.

FIG. 4 is a block diagram illustrating an example of a functional configuration of the medical arm system according to the embodiment.

FIG. 5 is a schematic perspective view for describing an overview of a technology regarding arm control based on setting of a virtual boundary in the medical arm system according to the embodiment.

FIG. 6 is an explanatory diagram for describing an overview of an example of a method of installing the virtual boundary according to the embodiment.

FIG. 7 is an explanatory diagram for describing an overview of an example of arm control in an arm system according to a comparative example.

FIG. 8 is a flowchart illustrating an example of a flow of a series of processing of the arm system according to the comparative example.

FIG. 9 is an explanatory diagram for describing an overview of arm control according to a first control example.

FIG. 10 is an explanatory diagram for describing an example of a method of setting a constraint point in the arm control according to the first control example.

FIG. 11 is a flowchart illustrating an example of a flow of a series of processing of the arm control according to the first control example.

FIG. 12 is an explanatory diagram for describing an overview of arm control according to a second control example.

FIG. 13 is a flowchart illustrating an example of a flow of a series of processing of the arm control according to the second control example.

FIG. 14 is an explanatory diagram for describing an overview of arm control according to a first example.

FIG. 15 is an explanatory diagram for describing an overview of an example of the arm control according to the first example.

FIG. 16 is an explanatory diagram for describing an overview of an example of the arm control according to the first example.

FIG. 17 is an explanatory diagram for describing an overview about a virtual boundary according to a first modification.

FIG. 18 is an explanatory diagram for describing an overview about a virtual boundary according to a second modification.

FIG. 19 is an explanatory diagram for describing an overview about a virtual boundary according to a third modification.

FIG. 20 is an explanatory diagram for describing an overview about a virtual boundary according to a fourth modification.

FIG. 21 is a functional block diagram illustrating a configuration example of a hardware configuration of an information processing apparatus according to the embodiment.

FIG. 22 is an explanatory diagram for describing an application of the medical arm system according to the embodiment.

### DESCRIPTION OF EMBODIMENTS

Favorable embodiments of the present disclosure will be described in detail with reference to the appended drawings. Note that, in the present specification and drawings, redundant description of configuration elements having substantially the same functional configuration is omitted by providing the same sign.

Note that the description will be given in the following order.

1. Overview of Medical Arm Device

1.1. Schematic Configuration of Medical Arm Device

1.2. Appearance of Medical Arm Device

1.3. Generalized Inverse Dynamics

1.4. Ideal Joint Control

2. Control of Medical Arm Device

2.1. Overview

2.2. Functional Configuration of Medical Arm System

2.3. Control Example of Medical Arm System

2.3.1. Basic Idea of Arm Control

2.3.2. Comparative Example: Operation Suppression Control

2.3.3. First Control Example: Operation Assist Control by Position Update of Constraint Point

2.3.4. Second Control Example: Operation Assist Control by Force Control

2.3.5. First Example: Operation Assist Control Example Using Virtual Boundary

2.3.6. Second Example: Operation Assist Control Example Using Virtual Boundary

2.4 Modification

2.4.1. First Modification

2.4.2. Second Modification

2.4.3. Third Modification

2.4.4. Fourth Modification

2.4.5. Supplement 3. Hardware Configuration

4. Application

5. Conclusion

### 1. OVERVIEW OF MEDICAL ARM DEVICE

### 1.1. Schematic Configuration of Medical Arm Device

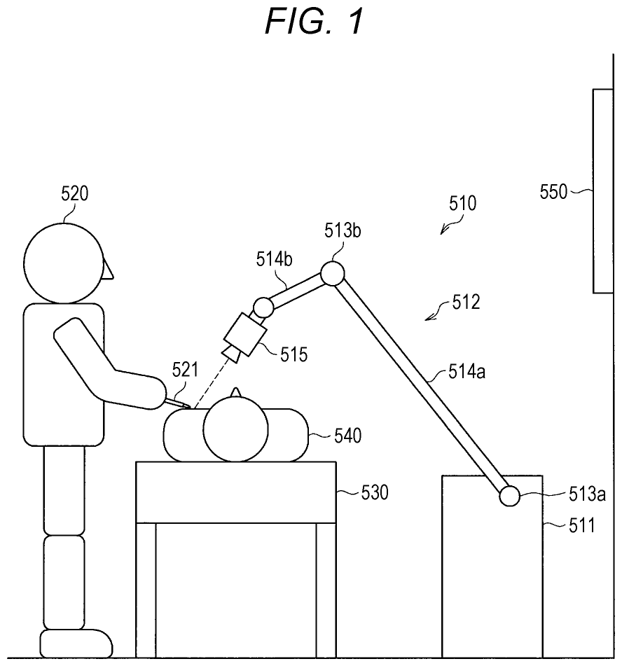

First, to make the present disclosure clearer, an example of a schematic configuration of a medical arm device will be described as an application of a case where the arm device according to an embodiment of the present disclosure is used for medical use. FIG. 1 is an explanatory diagram for describing an example of a schematic configuration of a medical arm device according to an embodiment of the present disclosure.

FIG. 1 schematically illustrates a state of an operation using the medical arm device according to the present embodiment. Specifically, referring to FIG. 1, a state in which a surgeon who is a practitioner (user) 520 is performing surgery for an operation target (patient) 540 on an operation table 530 using a surgical instrument 521 such as a scalpel, tweezers, or forceps, for example, is illustrated. Note that, in the following description, the term “operation” is a generic term for various types of medical treatment such as surgery and examination performed by a surgeon as the user 520 for the patient as the operation target 540 Furthermore, the example in FIG. 1 illustrates a state of surgery as an example of the operation, but the operation using the medical arm device 510 is not limited to surgery, and may be various operations such as an examination using an endoscope.

The medical arm device 510 according to the present embodiment is provided beside the operation table 530. The medical arm device 510 includes a base unit 511 that is a base, an arm unit 512 extending from the base unit 511, and an imaging unit 515 connected to a distal end of the arm unit 512 as a distal end unit The arm unit 512 includes a plurality of joint units 513a, 513b, and 513c, a plurality of links 514a and 514b connected by the joint units 513a and 513b, and the imaging unit 515 provided at the distal end of the arm unit 512. In the example illustrated in FIG. 1, the arm unit 512 includes the three joint units 513a to 513c and the two links 514a and 514b for the sake of simplicity. However, in reality, the numbers and shapes of the joint units 513a to 513c and the links 514a and 514b, the direction of drive shafts of the joint units 513a to 513c, and the like may be appropriately set to realize a desired degree of freedom in consideration of the degrees of freedom in the positions and postures of the arm unit 512 and the imaging unit 515.

The joint units 513a to 513c have a function to rotatably connect the links 514a and 514b to each other, and the drive of the arm unit 512 is controlled when the rotation of the joint units 513a to 513c is driven. Here, in the following description, the position of each configuration member of the medical arm device 510 means the position (coordinates) in the space defined for drive control, and the posture of each configuration member means the direction (angle) with respect to any axis in the space defined for drive control. Furthermore, in the following description, drive (or drive control) of the arm unit 512 refers to the position and posture of each configuration member of the arm unit 512 being changed (change being controlled) by drive (drive control) of the joint units 513a to 513c.

The imaging unit 515 is connected to the distal end of the arm unit 512 as the distal end unit. The imaging unit 515 is a unit that acquires an image of an imaging target, and is, for example, a camera that can capture a moving image or a still image or the like. As illustrated in FIG. 1, the positions and postures of the arm unit 512 and the imaging unit 515 are controlled by the medical arm device 510 so that the imaging unit 515 provided at the distal end of the arm unit 512 captures a state of the operation site of the operation target 540. Note that the configuration of the imaging unit 515 connected to the distal end of the arm unit 512 as the distal end unit is not particularly limited, and various medical instruments may be connected. Examples of the medical instruments include various units used in operations, such as an endoscope and microscope, units having an imaging function such as the above-described imaging unit 515, and various operation tools and examination devices. Furthermore, a stereo camera having two imaging units (camera units) may be provided at the distal end of the arm unit 512 and may capture an imaging target as a three-dimensional image (3D image). Note that the medical arm device 510 provided with a camera unit such as the imaging unit 515 or the stereo camera for capturing the operation site as the distal end unit is also referred to as video microscope (VM) arm device.

Furthermore, at a position facing the user 520, a display device 550 such as a monitor or a display is installed. An image of an operation site captured by the imaging unit 515 is displayed as an electronic image on a display screen of the display device 550. The user 520 performs various types of treatment while viewing the electronic image of the operation site displayed on the display screen of the display device 550.

Furthermore, a control device that controls the operation of the medical arm device 510 (for example, drive of the arm unit 512) may be separately provided, and a system including the medical arm device 510 and the control device may be configured. Note that, in the present disclosure, the term “medical arm system” can include both a case where the medical arm device 510 is configured to be operable alone and a case of a system including the medical arm device 510 and a control device of the medical arm device 510.

Thus, the present embodiment proposes, in the medical field, performing surgery while capturing an operation site by the medical arm device 510.

As an application of the case of using the medical arm device according to the present embodiment, an example of a case of using a surgical video microscope device provided with an arm as the medical arm device has been described with reference to FIG. 1.

### 1.2. Appearance of Medical Arm Device

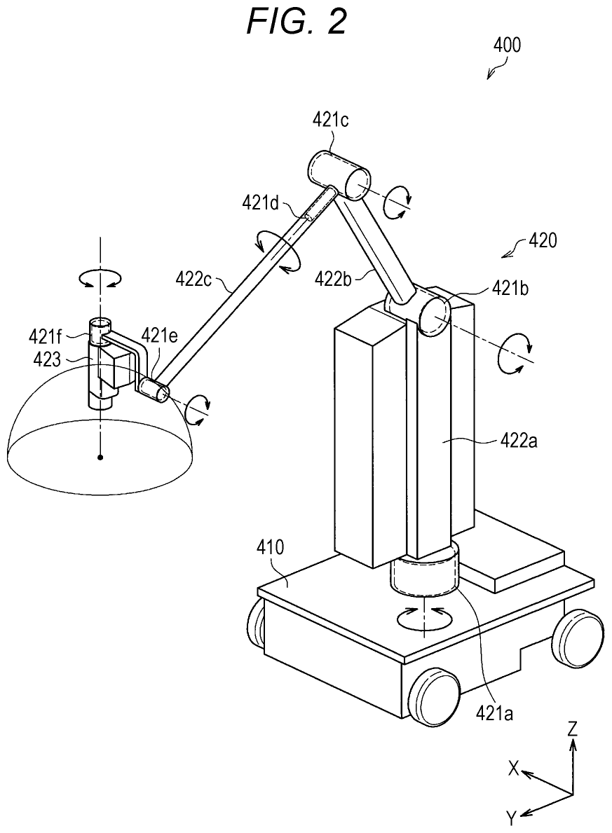

Next, a schematic configuration of the medical arm device according to an embodiment of the present disclosure will be described with reference to FIG. 2. FIG. 2 is a schematic view illustrating an appearance of the medical arm device according to an embodiment of the present disclosure.

Referring to FIG. 2, a medical arm device 400 according to the present embodiment includes a base unit 410 and an arm unit 420. The base unit 410 is a base of the medical arm device 400, and the arm unit 420 is extended from the base unit 410. Furthermore, although not illustrated in FIG. 2, a control unit that integrally controls the medical arm device 400 may be provided in the base unit 410, and drive of the arm unit 420 may be controlled by the control unit. The control unit is configured by, for example, various signal processing circuits such as a central processing unit (CPU) and a digital signal processor (DSP).

The arm unit 420 includes a plurality of joint units 421a to 421f, a plurality of links 422a to 422c mutually connected by the joint units 421a to 421f, and an imaging unit 423 provided at the distal end of the arm unit 420.

The links 422a to 422c are rod-like members, and one end of the link 422a is connected to the base unit 410 via the joint unit 421a, the other end of the link 422a is connected to one end of the link 422b via the joint unit 421b, and moreover, the other end of the link 422b is connected to one end of the link 422c via the joint units 421c and 421d. Moreover, the imaging unit 423 is connected to the distal end of the arm unit 420, in other words, the other end of the link 422c via the joint units 421e and 421f. As described above, the ends of the plurality of links 422a to 422c are connected one another by the joint units 421a to 421f with the base unit 410 as a fulcrum, so that an arm shape extended from the base unit 410 is configured.

The imaging unit 423 is a unit that acquires an image of an imaging target, and is, for example, a camera that captures a moving image or a still image or the like. When drive of the arm unit 420 is controlled, the position and posture of the imaging unit 423 are controlled. In the present embodiment, the imaging unit 423 captures a partial region of a body of a patient, which is an operation site, for example. Note that the distal end unit provided at the distal end of the arm unit 420 is not limited to the imaging unit 423, and various medical instruments may be connected to the distal end of the arm unit 420 as the distal end units.

Here, hereinafter, the medical arm device 400 will be described defining coordinate axes as illustrated in FIG. 2. Furthermore, an up-down direction, a front-back direction, and a right-left direction will be defined in accordance with the coordinate axes. In other words, the up-down direction with respect to the base unit 410 installed on a floor is defined as a z-axis direction and the up-down direction. Furthermore, a direction orthogonal to the z axis and in which the arm unit 420 is extended from the base unit 410 (in other words, a direction in which the imaging unit 423 is located with respect to the base unit 410) is defined as a y-axis direction and the front-back direction. Moreover, a direction orthogonal to the y axis and the z axis is defined as an x-axis direction and the right-left direction.

The joint units 421a to 421f rotatably connect the links 422a to 422c to one another. The joint units 421a to 421f include actuators, and have a rotation mechanism that is rotationally driven about a predetermined rotation axis by drive of the actuators. By controlling rotational drive of each of the joint units 421a to 421f, drive of the arm unit 420 such as extending or contracting (folding) of the arm unit 420 can be controlled. Here, the drive of the joint units 421a to 421f is controlled by whole body coordination control described in “1.3. Generalized Inverse Dynamics” below and by ideal joint control described in “1.4. Ideal Joint Control” below. Furthermore, as described above, since the joint units 421a to 421f have the rotation mechanism, in the following description, the drive control of the joint units 421a to 421f specifically means control of rotation angles and/or generated torque (torque generated by the joint units 421a to 4210 of the joint units 421a to 421f.

The medical arm device 400 according to the present embodiment includes the six joint units 421a to 421f and realizes six degrees of freedom with respect to the drive of the arm unit 420. Specifically, as illustrated in FIG. 2, the joint units 421a, 421d, and 421f are provided to have long axis directions of the connected links 422a to 422c and an imaging direction of the connected imaging unit 423 as rotation axis directions, and the joint units 421b, 421c, and 421e are provided to have the x-axis direction that is a direction of changing connection angles of the links 422a to 422c and the imaging unit 423 in a y-z plane (a plane defined by the y axis and the z axis) as the rotation axis directions. As described above, in the present embodiment, the joint units 421a, 421d, and 421f have a function to perform so-called yawing, and the joint units 421b, 421c, and 421e have a function to perform so-called pitching.

With such configuration of the arm unit 420, the medical arm device 400 according to the present embodiment realizes the six degrees of freedom with respect to the drive of the arm unit 420, thereby freely moving the imaging unit 423 within a movable range of the arm unit 420. FIG. 2 illustrates a hemisphere as an example of the movable range of the imaging unit 423. In a case where a central point of the hemisphere is a capture center of the operation site captured by the imaging unit 423, the operation site can be captured from various angles by moving the imaging unit 423 on a spherical surface of the hemisphere in a state where the capture center of the imaging unit 423 is fixed to the central point of the hemisphere.

### 1.3. Generalized Inverse Dynamics

Next, an overview of generalized inverse dynamics used for the whole body coordination control of the medical arm device 400 in the present embodiment will be described.

The generalized inverse dynamics is basic arithmetic operation in the whole body coordination control of a multilink structure configured by connecting a plurality of links by a plurality of joint units (for example, the arm unit 420 illustrated in FIG. 2 in the present embodiment), for converting motion purposes regarding various dimensions in various operation spaces into torque to be caused in the plurality of joint units in consideration of various constraint conditions.

The operation space is an important concept in force control of a robot device. The operation space is a space for describing a relationship between force acting on the multilink structure and acceleration of the multilink structure. When the drive control of the multilink structure is performed not by position control but by force control, the concept of the operation space is necessary in a case of using a contact between the multilink structure and an environment as a constraint condition. The operation space is, for example, a joint space, a Cartesian space, a momentum space, or the like, which is a space to which the multilink structure belongs.

The motion purpose represents a target value in the drive control of the multilink structure, and is, for example, a target value of a position, a speed, an acceleration, a force, an impedance, or the like of the multilink structure to be achieved by the drive control.

The constraint condition is a constraint condition regarding the position, speed, acceleration, force, or the like of the multilink structure, which is determined according to a shape or a structure of the multilink structure, an environment around the multilink structure, setting by the user, and the like. For example, the constraint condition includes information regarding a generated force, a priority, presence/absence of a non-drive joint, a vertical reaction force, a friction weight, a support polygon, and the like.

In the generalized dynamics, to establish both stability of numerical calculation and real time processing efficiency, an arithmetic algorithm includes a virtual force determination process (virtual force calculation processing) as a first stage and a real force conversion process (real force calculation processing) as a second stage. In the virtual force calculation processing as the first stage, a virtual force that is a virtual force necessary for achievement of each motion purpose and acting on the operation space is determined while considering the priority of the motion purpose and a maximum value of the virtual force. In the real force calculation processing as the second stage, the above-obtained virtual force is converted into a real force realizable in the actual configuration of the multilink structure, such as a joint force or an external force, while considering the constraints regarding the non-drive joint, the vertical reaction force, the friction weight, the support polygon, and the like. Hereinafter, the virtual force calculation processing and the real force calculation processing will be described in detail. Note that, in the description of the virtual force calculation processing and the real force calculation processing below, and the ideal joint control to be described below, description may be performed using the configuration of the arm unit 420 of the medical arm device 400 according to the present embodiment illustrated in FIG. 2 as a specific example, in order to facilitate understanding.

### 1.3.1. Virtual Force Calculation Processing

A vector configured by a certain physical quantity at each joint unit of the multilink structure is called generalized variable q (also referred to as a joint value q or a joint space q). An operation space x is defined by the following expression (1) using a time derivative value of the generalized variable q and the Jacobian J.

[Math. 1]

\dot{x}=J\dot{q} (1)

In the present embodiment, for example, q is a rotation angle of the joint units 421a to 421f of the arm unit 420. An equation of motion regarding the operation space x is described by the following expression (2).

[Math. 2]

\ddot{x}=Λ⁻¹f+c (2)

Here, f represents a force acting on the operation space x. Furthermore, Λ⁻¹ is an operation space inertia inverse matrix, and c is called operation space bias acceleration, which are respectively expressed by the following expressions (3) and (4).

[Math. 3]

Λ⁻¹=JH⁻¹J^{T} (3)

c=JH⁻¹(τ−b)+\dot{J}\dot{q} (4)

Note that H represents a joint space inertia matrix, τ represents a joint force corresponding to the joint value q (for example, the generated torque at the joint units 421a to 421f), and b represents gravity, a Coriolis force, and a centrifugal force.

In the generalized inverse dynamics, it is known that the motion purpose of the position and speed regarding the operation space x can be expressed as an acceleration of the operation space x. At this time, the virtual force fᵥ to act on the operation space x to realize an operation space acceleration that is a target value given as the motion purpose can be obtained by solving a kind of linear complementary problem (LCP) as in the expression (5) below according to the above expression (1).

$$[ Math . 4 ]$$

$$w + \ddot{x} = {Λ}^{- 1} {f}_{v} + c \\ s . t . \{ \begin{matrix}( ( {w}_{i} < 0 ) ⋂ ( {f}_{{v}_{i}} = {U}_{i} ) ) ⋃ \\ ( ( {w}_{i} > 0 ) ⋂ ( {f}_{{v}_{i}} = {L}_{i} ) ) ⋃ \\ ( ( {w}_{i} = 0 ) ⋂ ( {L}_{i} < {f}_{{v}_{i}} < {U}_{i} ) )\end{matrix} \qquad ( 5 )$$

Here, Lᵢ and Uᵢ respectively represent a negative lower limit value (including −∞) of an i-th component of and a positive upper limit value (including +∞) of the i-th component of fᵥ. The above LCP can be solved using, for example, an iterative method, a pivot method, a method applying robust acceleration control, or the like.

Note that the operation space inertia inverse matrix Λ⁻¹ and the bias acceleration c have a large calculation cost when calculated according to the expressions (3) and (4) that are defining expressions. Therefore, a method of calculating the processing of calculating the operation space inertia inverse matrix Λ⁻¹ at a high speed by applying a quasi-dynamics operation (FWD) for obtaining a generalized acceleration (joint acceleration) from the generalized force (joint force τ) of the multilink structure has been proposed. Specifically, the operation space inertia inverse matrix Λ⁻¹ and the bias acceleration c can be obtained from information regarding forces acting on the multilink structure (for example, of the arm unit 420 and the joint units 421a to 421f), such as the joint space q, the joint force τ, and the gravity g by using the forward dynamics arithmetic operation FWD. The operation space inertia inverse matrix Λ⁻¹ can be calculated with a calculation amount of O(N) with respect to the number N of the joint units by applying the forward dynamics arithmetic operation FWD regarding the operation space.

Here, as a setting example of the motion purpose, a condition for achieving the target value (expressed by adding a superscript bar to second order differentiation of x) of the operation space acceleration with a virtual force fᵥᵢ equal to or smaller than an absolute value Fᵢ can be expressed by the following expression (6).

[Math. 5]

Lᵢ=−Fᵢ,

Uᵢ=Fᵢ,

\ddot{x}ᵢ=\overline{\ddot{x}}ᵢ (6)

Furthermore, as described above, the motion purpose regarding the position and speed of the operation space x can be expressed as the target value of the operation space acceleration, and is specifically expressed by the following expression (7) (the target value of the position and speed of the operation space x is expressed by x and adding the superscript bar to first order differentiation of x).

[Math. 6]

\overline{\ddot{x}}ᵢ=Kₚ(x̅ᵢ−xᵢ)+Kᵥ(\overline{\dot{x}}ᵢ−\dot{x}ᵢ) (7)

In addition, by use of a concept of decomposition operation space, the motion purpose regarding an operation space (momentum, Cartesian relative coordinates, interlocking joint, or the like) expressed by a linear sum of other operation spaces can be set. Note that it is necessary to give priority to competing motion purposes. The above LCP can be solved for each priority in ascending order from a low priority, and the virtual force obtained by the LCP in the previous stage can be made to act as a known external force of the LCP in the next stage.

### 1.3.2. Real Force Calculation Processing

In the real force calculation processing as the second stage of the generalized inverse dynamics, processing of replacing the virtual force fᵥ obtained in the above (2-2-1. Virtual Force Determination Process) with real joint force and external force is performed. A condition for realizing the generalized force τ=Jᵥ^{T}fᵥ by the virtual force with generated torque τₐ generated in the joint unit and an external force fₑ is expressed by the following expression (8).

$$\begin{matrix}[ Math . 7 ] & \\ [ \begin{matrix}{J}_{v u}^{T} \\ {J}_{v a}^{T}\end{matrix} ] ( {f}_{v} - Δ {f}_{v} ) = [ \begin{matrix}{J}_{e u}^{T} \\ {J}_{e a}^{T}\end{matrix} ] {f}_{e} + [ \begin{matrix}0 \\ {τ}_{a}\end{matrix} ] & ( 8 )\end{matrix}$$

Here, the suffix a represents a set of drive joint units (drive joint set), and the suffix u represents a set of non-drive joint units (non-drive joint set). In other words, the upper part of the above expression (8) represents balance of the forces of the space (non-drive joint space) by the non-drive joint units, and the lower part represents balance of the forces of the space (drive joint space) by the drive joint units. Jᵥᵤ and Jᵥₐ are respectively a non-drive joint component and a drive joint component of the Jacobian regarding the operation space where the virtual force fᵥ acts. Jₑᵤ and Jₑₐ are a non-drive joint component and a drive joint component of the Jacobian regarding the operation space where the external force fₑ acts. Δfᵥ represents an unrealizable component with the real force, of the virtual force fᵥ.

The upper part of the expression (8) is undefined. For example, fₑ and Δfᵥ can be obtained by solving a quadratic programing problem (QP) as described in the following expression (9).

[Math. 8]

min ½ε^{T}Q₁ε+½ξ^{T}Q₂ξ

s.t. Uξ≥ν (9)

Here, ε is a difference between both sides of the upper part of the expression (8), and represents an equation error of the expression (8). ξ is a connected vector of fₑ and Δfᵥ and represents a variable vector. Q₁ and Q₂ are positive definite symmetric matrices that represent weights at minimization. Furthermore, inequality constraint of the expression (9) is used to express the constraint condition regarding the external force such as the vertical reaction force, friction cone, maximum value of the external force, or support polygon. For example, the inequality constraint regarding a rectangular support polygon is expressed by the following expression (10).

[Math. 9]

|Fₓ|≤μₜF_{z},

|F_{y}|≤μₜF_{z},

F_{z}≥0,

|Mₓ|≤d_{y}F_{z},

|M_{y}|≤dₓF_{z},

|M_{z}|≤μᵣF_{z} (10)

Here, z represents a normal direction of a contact surface, and x and y represent orthogonal two-tangent directions perpendicular to z. (Fₓ, F_{y}, F_{z}) and (Mₓ, M_{y}, M_{z}) represent an external force and an external force moment acting on a contact point. μₜ and μᵣ are friction coefficients regarding translation and rotation, respectively. (dₓ, d_{y}) represents the size of the support polygon.

From the above expressions (9) and (10), solutions fₑ and Δfᵥ of a minimum norm or a minimum error are obtained. By substituting fₑ and Δfᵥ obtained from the above expression (9) into the lower part of the above expression (8), the joint force τₐ necessary for realizing the motion purpose can be obtained.

In a case of a system where a base is fixed and there is no non-drive joint, all virtual forces can be replaced only with the joint force, and fₑ=0 and Δfᵥ=0 can be set in the above expression (8). In this case, the following expression (11) can be obtained for the joint force τₐ from the lower part of the above expression (8).

[Math. 10]

τₐ=Jᵥₐ^{T}fᵥ (11)

The whole body coordination control using the generalized inverse dynamics according to the present embodiment has been described. By sequentially performing the virtual force calculation processing and the real force calculation processing as described above, the joint force τₐ for achieving a desired motion purpose can be obtained. In other words, conversely speaking, by reflecting the calculated joint force τₐ in a theoretical model in the motion of the joint units 421a to 421f, the joint units 421a to 421f are driven to achieve the desired motion purpose.

Note that, regarding the whole body coordination control using the generalized inverse dynamics described so far, in particular, details of the process of deriving the virtual force fᵥ, the method of solving the LCP to obtain the virtual force fᵥ, the solution of the QP problem, and the like, reference can be made to JP 2009-95959A and JP 2010-188471A, which are prior patent applications filed by the present applicant, for example.

### 1.4. Ideal Joint Control

Next, the ideal joint control according to the present embodiment will be described. The motion of each of the joint units 421a to 421f is modeled by the equation of motion of the second order lag system of the following expression (12).

[Math. 11]

Iₐ\ddot{q}=τₐ+τₑ−νₐ\dot{q} (12)

Here, Iₐ represents moment of inertia (inertia) at the joint unit, τₐ represents the generated torque of the joint units 421a to 421f, τₑ represents external torque acting on each of the joint units 421a to 421f from the outside, and νₑ represents a viscous drag coefficient in each of the joint units 421a to 421f. The above expression (12) can also be said to be a theoretical model that represents the motion of the actuators 430 in the joint units 421a to 421f.

τₐ that is the real force to act on each of the joint units 421a to 421f for realizing the motion purpose can be calculated using the motion purpose and the constraint condition by the arithmetic operation using the generalized inverse dynamics described in “1.3. Generalized Inverse Dynamics” above. Therefore, ideally, by applying each calculated τₐ to the above expression (12), a response according to the theoretical model illustrated in the above expression (12) is realized, in other words, the desired motion purpose should be achieved.

However, in practice, errors (modeling errors) may occur between the motions of the joint units 421a to 421f and the theoretical model as illustrated in the above expression (12), due to the influence of various types of disturbance. The modeling errors can be roughly classified into those due to mass property such as weight, center of gravity, inertia tensor of the multilink structure, and those due to friction, inertia, and the like inside joint units 421a to 421f. Among them, the modeling errors due to the former mass property can be relatively easily reduced at the time of constructing the theoretical model by improving the accuracy of computer aided design (CAD) data and applying an identification method.

Meanwhile, the modeling errors due to the latter friction, inertia, and the like inside the joint units 421a to 421f are caused by phenomena that are difficult to model, such as friction in a reduction gear 426 of the joint units 421a to 421f, for example, and a modeling error that is not ignored may remain during theoretical model construction. Furthermore, there is a possibility that an error occurs between the values of the inertia Iₐ and the viscous drag coefficient νₑ in the above expression (12) and the values in the actual joint units 421a to 421f. These errors that are difficult to model can become the disturbance in the drive control of the joint units 421a to 421f. Therefore, in practice, the motions of the joint units 421a to 421f may not respond according to the theoretical model illustrated in the above expression (12), due to the influence of such disturbance. Therefore, even when the real force τₐ, which is a joint force calculated by the generalized inverse dynamics, is applied, there may be a case where the motion purpose that is the control target is not achieved. In the present embodiment, correcting the responses of the joint units 421a to 421f so as to perform ideal responses according to the theoretical model illustrated in the above expression (12), by adding an active control system to each of the joint units 421a to 421f, is considered. Specifically, in the present embodiment, not only performing friction compensation type torque control using the torque sensors 428 and 428a of the joint units 421a to 421f but also performing an ideal response according to the theoretical values up to the inertia Iₐ and the viscous drag coefficient νₐ to the necessary generated torque τₐ and external torque τₑ becomes possible.

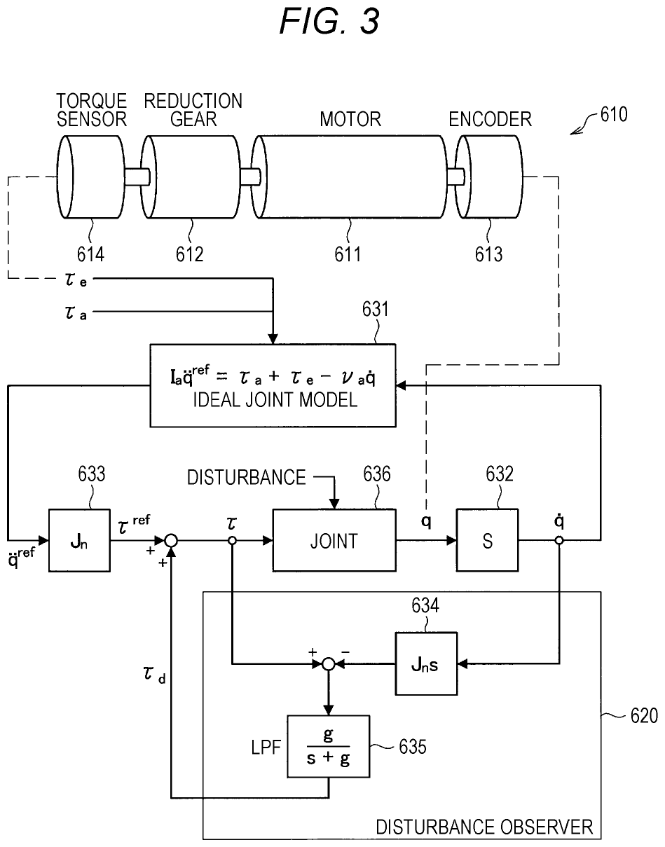

In the present embodiment, control of the drive of the joint units 421a to 421f of the medical arm device 400 to perform ideal responses as described in the above expression (12) is called ideal joint control. Here, in the following description, an actuator controlled to be driven by the ideal joint control is also referred to as a virtualized actuator (VA) because of performing an ideal response. Hereinafter, the ideal joint control according to the present embodiment will be described with reference to FIG. 3.

FIG. 3 is an explanatory diagram for describing the ideal joint control according to an embodiment of the present disclosure. Note that FIG. 3 schematically illustrates a conceptual arithmetic unit that performs various arithmetic operations regarding the ideal joint control in blocks.

An actuator 610 schematically illustrates the mechanism of the actuator that configures each joint unit of the arm unit. As illustrated in FIG. 3, the actuator 610 includes a motor (Motor) 611, a reduction gear (Reduction Gear) 612, an encoder (Encoder) 613, and a torque sensor (Torque Sensor) 614.

Here, a response of the actuator 610 according to the theoretical model expressed by the above expression (12) is nothing less than achievement of the rotation angular acceleration on the left side when the right side of the expression (12) is given. Furthermore, as illustrated in the above expression (12), the theoretical model includes an external torque term τₑ acting on the actuator 610. In the present embodiment, the external torque term τₑ is measured by the torque sensor 614 in order to perform the ideal joint control. Furthermore, a disturbance observer 620 is applied to calculate a disturbance estimation value τ_{d} that is an estimation value of a torque due to disturbance on the basis of a rotation angle q of the actuator 610 measured by the encoder 613.

A block 631 represents an arithmetic unit that performs an arithmetic operation according to an ideal joint model of the joint units 421a to 421f illustrated in the above expression (12). The block 631 can output a rotation angular acceleration target value (a second order differentiation of a rotation angle target value q^{ref}) described on the left side of the above expression (12), using the generated torque τₐ, the external torque τₑ, and the rotation angular speed (first order differentiation of the rotation angle q) as inputs.

In the present embodiment, the generated torque τₐ calculated by the method described in “1.3. Generalized inverse dynamics” above and the external torque τₑ measured by the torque sensor 614 are input to the block 631. Meanwhile, when the rotation angle q measured by the encoder 613 is input to a block 632 representing an arithmetic unit that performs a differential operation, the rotation angular speed (the first order differentiation of the rotation angle q) is calculated. When the rotation angular speed calculated in the block 632 is input to the block 631 in addition to the generated torque τₐ and the external torque τₑ, the rotation angular acceleration target value is calculated by the block 631. The calculated rotation angular acceleration target value is input to a block 633.

The block 633 represents an arithmetic unit that calculates a torque generated in the actuator 610 on the basis of the rotation angular acceleration of the actuator 610. In the present embodiment, specifically, the block 633 can obtain a torque target value τ^{ref} by multiplying the rotation angular acceleration target value by nominal inertia in the actuator 610. In the ideal response, the desired motion purpose should be achieved by causing the actuator 610 to generate the torque target value τ^{ref}. However, as described above, there is a case where the influence of the disturbance or the like occurs in the actual response. Therefore, in the present embodiment, the disturbance observer 620 calculates the disturbance estimation value τ_{d} and corrects the torque target value τ^{ref} using the disturbance estimation value τ_{d}.

A configuration of the disturbance observer 620 will be described. As illustrated in FIG. 3, the disturbance observer 620 calculates the disturbance estimation value τ_{d} on the basis of the torque command value τ and the rotation angular speed output from the rotation angle q measured by the encoder 613. Here, the torque command value τ is a torque value to be finally generated in the actuator 610 after the influence of disturbance is corrected. For example, in a case where the disturbance estimation value τ_{d} is not calculated, the torque command value τ becomes the torque target value τ^{ref}.

The disturbance observer 620 includes a block 634 and a block 635. The block 634 represents an arithmetic unit that calculates a torque generated in the actuator 610 on the basis of the rotation angular speed of the actuator 610. In the present embodiment, specifically, the rotation angular speed calculated by the block 632 from the rotation angle q measured by the encoder 613 is input to the block 634. The block 634 obtains the rotation angular acceleration by performing an arithmetic operation represented by a transfer function Jₙs, in other words, by differentiating the rotation angular speed, and further multiplies the calculated rotation angular acceleration by the nominal inertia Jₙ, thereby calculating an estimation value of the torque actually acting on the actuator 610 (torque estimation value).

In the disturbance observer 620, the difference between the torque estimation value and the torque command value τ is obtained, whereby the disturbance estimation value τ_{d}, which is the value of the torque due to the disturbance, is estimated. Specifically, the disturbance estimation value τ_{d} may be a difference between the torque command value τ in the control of the preceding cycle and the torque estimation value in the current control. Since the torque estimation value calculated by the block 634 is based on the actual measurement value and the torque command value τ calculated by the block 633 is based on the ideal theoretical model of the joint units 421a to 421f illustrated in the block 631, the influence of the disturbance, which is not considered in the theoretical model, can be estimated by taking the difference between the torque estimation value and the torque command value τ.

Furthermore, the disturbance observer 620 is provided with a low pass filter (LPF) illustrated in a block 635 to prevent system divergence. The block 635 outputs only a low frequency component to the input value by performing an arithmetic operation represented by a transfer function g/(s+g) to stabilize the system. In the present embodiment, the difference value between the torque estimation value and the torque command value τ^{ref} calculated by the block 634 is input to the block 635, and a low frequency component of the difference value is calculated as the disturbance estimation value τ_{d}.

In the present embodiment, feedforward control to add the disturbance estimation value τ_{d} calculated by the disturbance observer 620 to the torque target value τ^{ref} is performed, whereby the torque command value τ that is the torque value to be finally generated in the actuator 610 is calculated. Then, the actuator 610 is driven on the basis of the torque command value τ. Specifically, the torque command value τ is converted into a corresponding current value (current command value), and the current command value is applied to the motor 611, so that the actuator 610 is driven.

As described above, with the configuration described with reference to FIG. 3, the response of the actuator 610 can be made to follow the target value even in a case where there is a disturbance component such as friction in the drive control of the joint units 421a to 421f according to the present embodiment. Furthermore, with regard to the drive control of the joint units 421a to 421f, an ideal response according to the inertia Iₐ and the viscous drag coefficient νₐ assumed by the theoretical model can be made.

Note that, for details of the above-described ideal joint control, JP 2009-269102A, which is a prior patent application filed by the present applicant, can be referred to, for example.

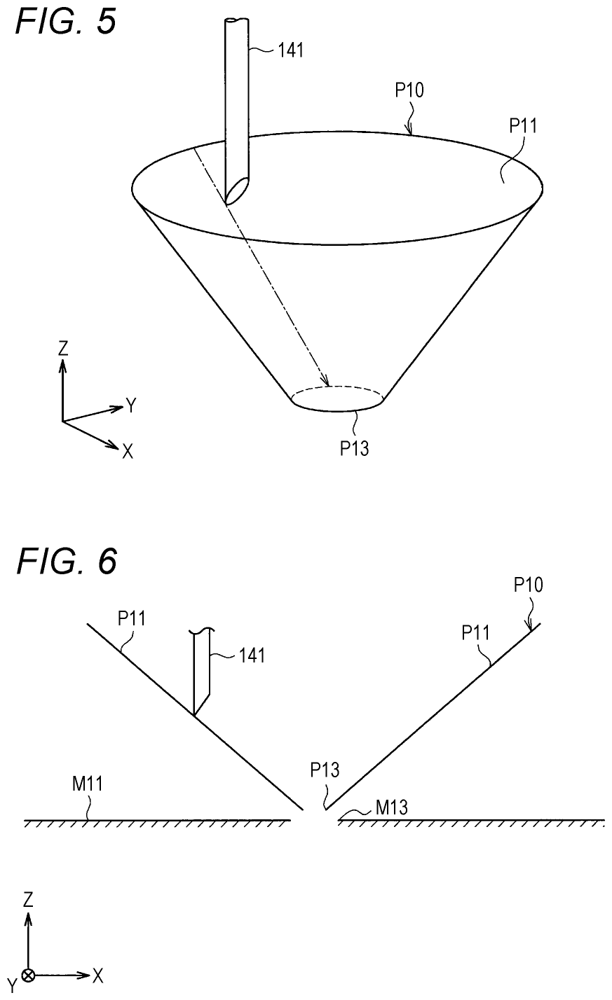

The generalized inverse dynamics used in the present embodiment has been described, and the ideal joint control according to the present embodiment has been described with reference to FIG. 3. As described above, in the present embodiment, the whole body coordination control, in which the drive parameters of the joint units 421a to 421f (for example, the generated torque values of the joint units 421a to 421f) for achieving the motion purpose of the arm unit 420 are calculated in consideration of the constraint condition, is performed using the generalized inverse dynamics. Furthermore, as described with reference to FIG. 5, in the present embodiment, the ideal joint control that realizes the ideal response based on the theoretical model in the drive control of the joint units 421a to 421f by performing correction of the generated torque value, which has been calculated in the whole body coordination control using the generalized inverse dynamics, in consideration of the influence of the disturbance, is performed. Therefore, in the present embodiment, highly accurate drive control that achieves the motion purpose becomes possible with regard to the drive of the arm unit 420.

### 2. CONTROL OF MEDICAL ARM DEVICE

Next, a technology regarding control of the medical arm device in the medical arm system according to an embodiment of the present disclosure will be described.

### 2.1. Overview

First, an overview of the technology regarding control of the medical arm device in the medical arm system according to an embodiment of the present disclosure will be described. In the medical arm system according to the present embodiment, virtual boundary surfaces (hereinafter also referred to as “virtual boundaries”) referred to as a virtual barrier and a virtual wall are set in a real space. Under such setting, in the medical arm system according to the present embodiment, the operation of the arm unit is controlled according to a positional relationship between the virtual boundary and the distal end unit held at the distal end of the arm unit. Specifically, a situation as if the virtual boundary existed in the real space is simulated on the basis of the control of the arm unit based on the whole body coordination control using the above-described generalized inverse dynamics.

### 2.2. Functional Configuration of Medical Arm System

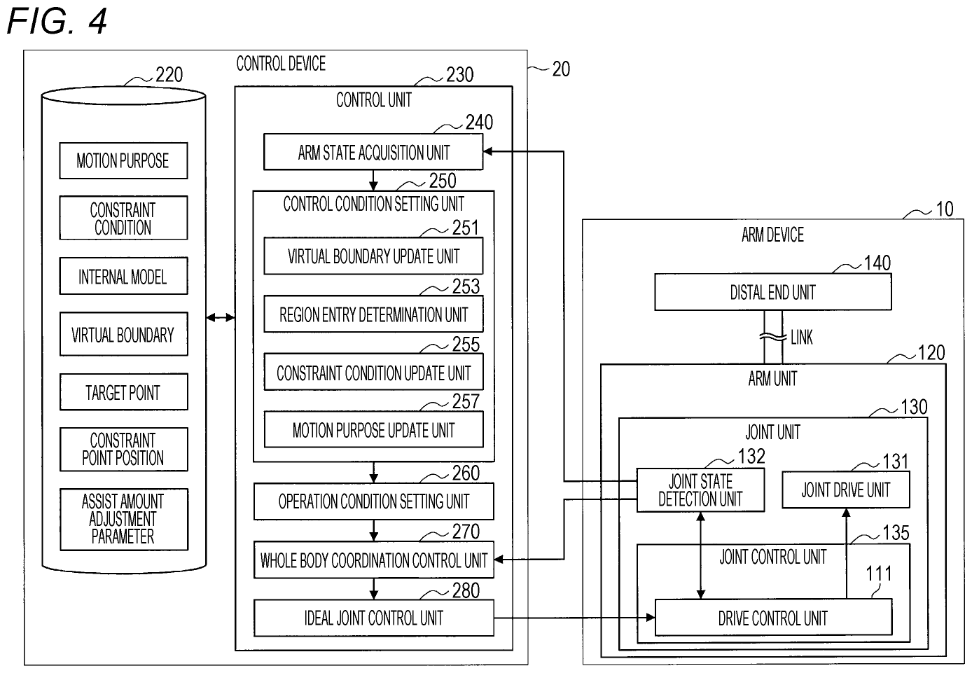

Here, an example of a functional configuration of the medical arm system according to an embodiment of the present disclosure will be described. In the medical arm system according to the present embodiment, drive of the plurality of joint units provided in the medical arm device is controlled on the basis of the whole body coordination control using the above-described generalized inverse dynamics, for example. For example, FIG. 4 is a block diagram illustrating a functional configuration of the medical arm system according to an embodiment of the present disclosure. Note that, in the robot arm control system illustrated in FIG. 4, a configuration related to drive control of an arm unit of a robot arm device will be mainly illustrated.

As illustrated in FIG. 4, a medical arm system 1 according to an embodiment of the present disclosure includes an arm device 10 and a control device 20. In the present embodiment, the control device 20 performs various arithmetic operations in the whole body coordination control described in “1.3. Generalized Inverse Dynamics” and the ideal joint control described in “1.4. Ideal Joint Control” above, and drive of the arm unit of the arm device 10 is controlled on the basis of an arithmetic operation result. Furthermore, a distal end unit 140 described below is held by the arm unit of the arm device 10. Hereinafter, configurations of the arm device 10 and the control device 20 will be described in detail.

The arm device 10 includes the arm unit that is a multilink structure including a plurality of joint units and a plurality of links, and drives the arm unit within a movable range to control the position and posture of the distal end unit provided at the distal end of the arm unit. The arm device 10 corresponds to the medical arm device 400 illustrated in FIG. 2.

As illustrated in FIG. 4, the arm device 10 includes an arm unit 120 and the distal end unit 140 held at a distal end of the arm unit 120.

The arm unit 120 is a multilink structure including a plurality of joint units and a plurality of links. The arm unit 120 corresponds to the arm unit 420 illustrated in FIG. 2. The arm unit 120 includes a joint unit 130. Note that, since functions and structures of the plurality of joint units included in the arm unit 120 are similar to one another, FIG. 4 illustrates a configuration of one joint unit 130 as a representative of the plurality of joint units.

The joint unit 130 rotatably connects the links with each other in the arm unit 120, and drives the arm unit 120 as rotational drive of the joint unit 130 is controlled by the control of the arm control unit 110. The joint unit 130 corresponds to the joint units 421a to 421f illustrated in FIG. 2. Furthermore, the joint unit 130 includes an actuator.

The joint unit 130 includes a joint drive unit 131, a joint state detection unit 132, and a joint control unit 135.

The joint control unit 135 controls drive of the joint unit 130 such that the arm device 10 is controlled in an integrated manner. Specifically, the joint control unit 135 includes a drive control unit 111. Drive of the joint unit 130 is controlled by the control of the drive control unit 111, so that the drive of the arm unit 120 is controlled. More specifically, the drive control unit 111 controls a current amount to be supplied to a motor in an actuator of the joint unit 130 to control the number of rotations of the motor, thereby controlling a rotation angle and generated torque in the joint unit 130. However, as described above, the drive control of the arm unit 120 by the drive control unit 111 is performed on the basis of the arithmetic operation result in the control device 20. Therefore, the current amount to be supplied to the motor in the actuator of the joint unit 130, which is controlled by the drive control unit 111, is a current amount determined on the basis of the arithmetic operation result in the control device 20.

The joint drive unit 131 is a drive mechanism in the actuator of the joint unit 130, and the joint unit 130 is rotationally driven as the joint drive unit 131 is driven. The drive of the joint drive unit 131 is controlled by the drive control unit 111. For example, the joint drive unit 131 has a configuration corresponding to, for example, a motor and a motor driver. In other words, the joint drive unit 131 being driven corresponds to the motor driver driving the motor with the current amount according to a command from the drive control unit 111.

The joint state detection unit 132 detects a state of the joint unit 130. Here, the state of the joint unit 130 may mean a state of motion of the joint unit 130. For example, the state of the joint unit 130 includes information regarding rotation of the joint unit 130, for example, information of the rotation angle, rotation angular speed, rotation angular acceleration, generated torque, and the like. In the present embodiment, the joint state detection unit 132 detects the rotation angle of the joint unit 130 and the generated torque and external torque of the joint unit 130 as the state of the joint unit 130. Note that the detection of the rotation angle q of the joint unit 130 and the detection of the generated torque and external torque of the joint unit 130 can be realized by an encoder and a torque sensor for detecting the state of the actuator. The joint state detection unit 132 transmits the detected state of the joint unit 130 to the control device 20.

The distal end unit 140 schematically illustrates a unit held at the distal end of the arm unit 120. Note that, in the present embodiment, various medical instruments can be connected to the distal end of the arm unit 120 as the distal end unit 140. Examples of the medical instruments include various operation tools such as a scalpel and forceps, and various units used in operation, such as a unit of various detection devices such as probes of an ultrasonic examination device. Furthermore, as another example, a unit having an imaging function such as an endoscope or a microscope may also be included in the medical instruments. Thus, the arm device 10 according to the present embodiment can be said to be a medical arm device provided with medical instruments. Note that the arm device 10 illustrated in FIG. 4 can also include the unit having an imaging function as the distal end unit, and a stereo camera having two imaging units (camera units) may be provided and captures the imaging target to be displayed as a 3D image.

The functional configuration of the arm device 10 has been described above. Next, a functional configuration of the control device 20 will be described. As illustrated in FIG. 4, the control device 20 includes a storage unit 220 and a control unit 230. Furthermore, although not illustrated in FIG. 4, the control device 20 may include an input unit for inputting various types of information, an output unit for outputting various types of information, and the like.

The control unit 230 integrally controls the control device 20 and performs various arithmetic operations for controlling the drive of the arm unit 120 in the arm device 10. Specifically, the control unit 230 sets a control condition of the operation of the arm unit 120 according to a positional relationship between the virtual boundary set to the real space and the distal end unit 140 held by the arm unit 120 of the arm device 10. Then, the control unit 230 performs various arithmetic operations in the whole body coordination control and the ideal joint control to control the drive of the arm unit 120 on the basis of the control condition. Hereinafter, the functional configuration of the control unit 230 will be described in detail. Since the whole body coordination control and the ideal joint control have been already described, detailed description is omitted here.

The control unit 230 includes an arm state acquisition unit 240, a control condition setting unit 250, an arithmetic condition setting unit 260, a whole body coordination control unit 270, and an ideal joint control unit 280. Furthermore, the control condition setting unit 250 includes a virtual boundary update unit 251, a region entry determination unit 253, a constraint condition update unit 255, and a motion purpose update unit 257.

The arm state acquisition unit 240 acquires the state (arm state) of the arm unit 120 on the basis of the state of the joint unit 130 detected by the joint state detection unit 132. Here, the arm state may mean the state of motion of the arm unit 120. For example, the arm state includes information such as the position, speed, acceleration, and force of the arm unit 120. As described above, the joint state detection unit 132 acquires, as the state of the joint unit 130, the information regarding the rotation of each joint unit 130, for example, the information of the rotation angle, rotation angular speed, rotation angular acceleration, generated torque, and the like. Furthermore, although to be described below, the storage unit 220 stores various types of information to be processed by the control device 20. In the present embodiment, the storage unit 220 may store various types of information (arm information) regarding the arm unit 120, for example, information defining the structure of the arm unit 120, in other words, the numbers of joint units 130 and links configuring the arm unit 120, connection situations between the links and the joint units 130, and lengths of the links, and the like. The arm state acquisition unit 240 can acquire the arm information from the storage unit 220. Therefore, the arm state acquisition unit 240 can acquire, as the arm state, information such as the positions (coordinates) in the space of the plurality of joint units 130, the plurality of links, and the distal end unit 140, and the forces acting on the joint units 130, the links, and the distal end unit 140, on the basis of the state and the arm information of the joint units 130. The arm state acquisition unit 240 outputs the acquired arm information to the control condition setting unit 250.

The virtual boundary update unit 251 sets and updates the virtual boundary on the basis of various conditions. For example, the storage unit 220 described below may store various types of information regarding the virtual boundary such as the shape and size of the virtual boundary (in other words, information regarding setting of the virtual boundary). The virtual boundary update unit 251 can acquire the information regarding the virtual boundary from the storage unit 220. Therefore, the virtual boundary update unit 251 can set and update the virtual boundary on the basis of the information regarding the virtual boundary. As a specific example, the virtual boundary update unit 251 may set and update the shape of the virtual boundary, the size of the virtual boundary, the position and posture of the virtual boundary in the real space, and the like.

For example, the virtual boundary update unit 251 may set the shape and the size of the virtual boundary as initial setting. In other words, the shape and size of the virtual boundary may be preset (in other words, may be determined before surgery). Since the shape, size, and the like of the virtual boundary are preset as described above, the user can obtain the same operational feeling every time, for example, and thus functions and effects such as improvement of procedure and improvement of safety can be expected.

Furthermore, the virtual boundary update unit 251 can update the virtual boundary (update the shape and the like of the virtual boundary, for example) in response to the operation of the arm unit 120 by the user. As a specific example, the virtual boundary update unit 251 can update the position, shape, and the like of the virtual boundary together with update of a target point regarding assist of movement of the distal end unit 140 held by the arm unit 120 at the time of the operation of the arm unit 120 by the user based on a function so-called position memory function (a function to store the position and posture of the arm in the space and enable the arm to return to the same position and posture again). Furthermore, as another example, the virtual boundary update unit 251 may set and update the virtual boundary in response to an instruction from the user via a predetermined input unit (illustration is omitted).

Furthermore, the virtual boundary update unit 251 may set and update the virtual boundary on the basis of a detection result of an object by a detector such as various sensors, a recognition result of an object according to an imaging result by an imaging unit, or the like. In other words, the virtual boundary update unit 251 may set and update the virtual boundary according to detection results of various states. As a specific example, the virtual boundary update unit 251 may set and update the position, posture, shape, size, and the like of the virtual boundary according to the detection result by the detector, or the like. Such control enables setting of the virtual boundary in a favorable manner according to a situation during surgery. Therefore, the setting and update of the virtual boundary can also be adaptively performed to avoid a contact between the distal end unit held by the arm unit and an object in the real space, for example.

Furthermore, the virtual boundary update unit 251 may set and update the virtual boundary according to the distal end unit held by the arm unit 120. As a specific example, the virtual boundary update unit 251 may set and update the position, posture, shape, size, and the like of the virtual boundary such that the virtual boundary is set in a favorable manner for assisting the procedure using the distal end unit according to the distal end unit (for example, a medical instrument) held by the arm unit 120. Furthermore, in a case where the distal end unit held by the arm unit 120 is changed, the virtual boundary update unit 251 may set and update the virtual boundary according to the distal end unit after change.

Of course, the above description is merely examples, and the method of setting and updating the virtual boundary is not particularly limited.

The region entry determination unit 253 determines entry of a point of action set using at least a part of the arm unit 120 as a base point into a region separated by the virtual boundary on the basis of the result of the setting and update of the virtual boundary and the arm information. As a specific example, the region entry determination unit 253 may recognize the position of the point of action as a relative position relative to a part of the arm unit 120 on the basis of the information of the position, posture, shape, and the like of the joint units 130 and the links configuring the arm unit 120. Furthermore, at this time, the region entry determination unit 253 may set the point of action at a position corresponding to a part (for example, the distal end or the like) of the distal end unit 140 by taking into account the position, posture, shape, and the like of the distal end unit 140 held by the arm unit 120. Then, the region entry determination unit 253 determines a contact between the virtual boundary and the point of action (in other words, determines the point of action being located on the virtual boundary) and determines whether or not the point of action enters at least one of a first region or a second region separated by the virtual boundary on the basis of a relative positional relationship between the virtual boundary and the point of action (for example, the distal end of the distal end unit 140).

Note that the point of action may be set after taking into account the position, posture, shape, and the like of the distal end unit 140 that can be held by the arm unit 120 regardless of whether or not the distal end unit 140 is actually held by the arm unit 120. Thereby, the state where the distal end unit 140 is held by the arm unit 120 can be virtually simulated even in a state where the distal end unit 140 is not held by the arm unit 120, for example. The point of action is also referred to as the ‘predetermined point’ elsewhere herein.

The constraint condition update unit 255 sets and updates a constraint condition regarding the control of the operation of the arm unit 120. Specifically, the constraint condition may be various types of information that restricts (constrains) the motion of the arm unit 120. More specifically, the constraint condition may be coordinates of a region where each configuration member of the arm unit is unmovable, an unmovable speed, a value of acceleration, a value of an ungenerable force, and the like. Furthermore, restriction ranges of various physical quantities under the constraint condition may be set according to inability to structurally realize the arm unit 120 or may be appropriately set by the user. The constraint condition update unit 255 according to the present embodiment may set and update the constraint condition according to a relationship between the virtual boundary and the point of action (for example, a relationship of relative positions and postures, or the like). As a specific example, in a case where the constraint condition update unit 255 determines that the point of action enters a region separated by the virtual boundary, the constraint condition update unit 255 may set and update the constraint condition for suppressing at least a part of the operation of the arm unit 120 to suppress the entry. Furthermore, in a case where the constraint condition update unit 255 determines that the point of action does not enter the region separated by the virtual boundary, the constraint condition update unit 255 may set and update the constraint condition such that the operation of the arm unit 120 is not suppressed. Note that processing of setting and updating the constraint condition and the control of the operation of the arm unit 120 according to the constraint condition will be separately described below in detail together with a more specific example.

The motion purpose update unit 257 sets and updates a motion condition regarding the control of the operation of the arm unit 120. Specifically, the motion purpose may be target values of the position and posture (coordinates), speed, acceleration, force, and the like of the distal end unit 140, or target values of the positions (coordinates), speeds, accelerations, forces, and the like of the plurality of joint units 130 and the plurality of links of the arm unit 120. The motion purpose update unit 257 according to the present embodiment may set and update the motion condition according to the relationship between the virtual boundary and the point of action. As a specific example, in a case where the motion purpose update unit 257 determines that the point of action enters the region separated by the virtual boundary, the motion purpose update unit 257 may set and update the motion purpose for causing a reaction force to work to suppress the entry. Note that processing of setting and updating the motion purpose and the control of the operation of the arm unit 120 according to the motion purpose will be separately described below in detail together with a more specific example.

The arithmetic condition setting unit 260 sets arithmetic operation conditions in an arithmetic operation regarding the whole body coordination control using the generalized inverse dynamics. Here, the arithmetic operation conditions may be the above-described motion purpose and constraint condition. The motion purpose may be various types of information regarding the motion of the arm unit 120. Furthermore, the arithmetic condition setting unit 260 includes a physical model for the structure of the arm unit 120 (in which, for example, the number and lengths of the links configuring the arm unit 120, the connection states of the links via the joint units 130, the movable ranges of the joint units 130, and the like are modeled), and may set a motion condition and the constraint condition by generating a control model in which the desired motion condition and constraint condition are reflected in the physical model.

Appropriate setting of the motion purpose and constraint condition enables the arm unit 120 to perform a desired operation. For example, as the motion purpose, not only can the distal end unit 140 be moved to a target position by setting a target value of the position of the distal end unit 140 but also the arm unit 120 can be driven by providing a constraint of movement by the constraint condition to prevent the arm unit 120 from intruding into a predetermined region in the space. In particular, in the present embodiment, as described above, the constraint condition and the motion purpose may be set or updated by the control condition setting unit 250 according to the setting of the virtual boundary and the positional relationship between the virtual boundary and the point of action (for example, the distal end of the distal end unit 140).

A specific example of the motion purpose may be an operation to suppress the entry of the distal end unit 140 to the region separated by the virtual boundary.

Furthermore, as another example, the motion purpose may be content to control the generated torque in each joint unit 130. Specifically, the motion purpose may be a power assist operation to control the state of the joint unit 130 to cancel the gravity acting on the arm unit 120, and further control the state of the joint unit 130 to support the movement of the arm unit 120 in a direction of a force provided from the outside. More specifically, in the power assist operation, the drive of each joint unit 130 is controlled to cause each joint unit 130 to generate a generated torque that cancels the external torque due to the gravity in each joint unit 130 of the arm unit 120, whereby the position and posture of the arm unit 120 are held in a predetermined state. In a case where an external torque is further added from the outside (for example, from the user) in the aforementioned state, the drive of each joint unit 130 is controlled to cause each joint unit 130 to generate a generated torque in the same direction as the added external torque. By performing such a power assist operation, the user can move the arm unit 120 with a smaller force in a case where the user manually moves the arm unit 120. Therefore, a feeling as if the user were moving the arm unit 120 under weightlessness can be provided to the user. Furthermore, the operation regarding suppression of the entry of the distal end unit 140 into the region separated by the virtual boundary and the power assist operation can be combined.

Note that, in the present embodiment, the motion purpose may mean an operation (motion) of the arm unit 120 realized by the whole body coordination control or may mean an instantaneous motion purpose in the operation (in other words, a target value in the motion purpose). For example, in the above-described power assist operation, performing the power assist operation to support the movement of the arm unit 120 in the direction of the force applied from the outside itself is the motion purpose. In the act of performing the power assist operation, the value of the generated torque in the same direction as the external torque applied to each joint unit 130 is set as the instantaneous motion purpose (the target value in the motion purpose). The motion purpose in the present embodiment is a concept including both the instantaneous motion purpose (for example, the target values of the positions, speeds, forces, and the like of the configuration members of the arm unit 120 at a certain time) and the operations of the configuration members of the arm unit 120 realized over time as a result of the instantaneous motion purpose having been continuously achieved. The instantaneous motion purpose is set each time in each step in an arithmetic operation for the whole body coordination control in the whole body coordination control unit 270, and the arithmetic operation is repeatedly performed, so that the desired motion purpose is finally achieved.

Furthermore, the viscous drag coefficient in a rotation motion of each joint unit 130 may be appropriately set when the motion purpose is set. The joint unit 130 according to the present embodiment is configured to be able to appropriately adjust the viscous drag coefficient in the rotation motion of the actuator. Therefore, by setting the viscous drag coefficient in the rotation motion of each joint unit 130 when setting the motion purpose, an easily rotatable state or a less easily rotatable state can be realized for the force applied from the outside, for example. As a specific example, in the above-described power assist operation, when the viscous drag coefficient in the joint unit 130 is set to be small, a force used by the user to move the arm unit 120 can be made smaller, and a weightless feeling provided to the user can be promoted. As described above, the viscous drag coefficient in the rotation motion of each joint unit 130 may be appropriately set according to the content of the motion purpose.

The whole body coordination control unit 270 calculates a control command value for the whole body coordination control by an arithmetic operation using the generalized inverse dynamics described with reference to FIG. 3.

The ideal joint control unit 280 calculates a command value for controlling the operation of the arm unit 120 to be finally transmitted to the arm device 10. Specifically, the ideal joint control unit 280 calculates the disturbance estimation value τ_{d} on the basis of the torque command value τ and the rotation angular speed calculated from the rotation angle q of the joint unit 130 detected by the joint state detection unit 132. Note that the torque command value τ mentioned here can correspond to a command value that represents the generated torque in the arm unit 120 to be finally transmitted to the arm device 10. Furthermore, the ideal joint control unit 280 calculates the torque command value τ that is a command value representing the torque to be generated in the arm unit 120 and finally transmitted to the arm device 10, using the disturbance estimation value τ_{d}. Specifically, the ideal joint control unit 280 adds the disturbance estimation value τ_{d} to τ^{ref} calculated from the ideal model of the joint unit 130 described in the above expression (12) to calculate the torque command value τ. For example, in a case where the disturbance estimation value τ_{d} is not calculated, the torque command value τ becomes the torque target value τ^{ref}.

The ideal joint control unit 280 transmits the calculated torque command value τ to the drive control unit 111 of the arm device 10. The drive control unit 111 performs control to supply the current amount corresponding to the transmitted torque command value τ to the motor in the actuator of the joint unit 130, thereby controlling the number of rotations of the motor and controlling the rotation angle and the generated torque in the joint unit 130.

In the medical arm system 1 according to the present embodiment, the drive control of the arm unit 120 in the arm device 10 is continuously performed during work using the arm unit 120, so the above-described processing in the arm device 10 and the control device 20 is repeatedly performed. In other words, the state of the joint unit 130 is detected by the joint state detection unit 132 of the arm device 10 and transmitted to the control device 20. The control device 20 performs various arithmetic operations regarding the whole body coordination control and the ideal joint control for controlling the drive of the arm unit 120 on the basis of the state of the joint unit 130, and the motion purpose and the constraint condition, and transmits the torque command value τ as the arithmetic operation result to the arm device 10. The arm device 10 controls the drive of the arm unit 120 on the basis of the torque command value τ, and the state of the joint unit 130 during or after the driving is detected by the joint state detection unit 132 again.

Description about other configurations included in the control device 20 will be continued.

The storage unit 220 stores various types of information processed by the control device 20. In the present embodiment, the storage unit 220 can store various parameters used for setting and updating of the virtual boundary. As a specific example, the storage unit 220 may store parameters such as the shape and size of the virtual boundary.

Furthermore, the storage unit 220 can store various parameters used in the arithmetic operation regarding the whole body coordination control and the ideal joint control performed by the control unit 230. For example, the storage unit 220 may store the motion purpose and the constraint condition used in the arithmetic operation regarding the whole body coordination control by the whole body coordination control unit 270. The motion purpose stored in the storage unit 220 may be, as described above, a motion purpose that can be set in advance, such as, for example, the distal end unit 140 standing still at a predetermined point in the space. Furthermore, the constraint conditions may be set in advance by the user and stored in the storage unit 220 according to a geometric configuration of the arm unit 120, the application of the arm device 10, and the like. Furthermore, the storage unit 220 may also store various types of information regarding the arm unit 120 used when the arm state acquisition unit 240 acquires the arm state. Moreover, the storage unit 220 may store the arithmetic operation result in the arithmetic operation regarding the whole body coordination control and the ideal joint control by the control unit 230, various numerical values calculated in the arithmetic operation process, and the like. As described above, the storage unit 220 may store any parameters regarding the various types of processing performed by the control unit 230, and the control unit 230 can performs various types of processing while mutually exchanging information with the storage unit 220.

Furthermore, the storage unit 220 may be used as a storage region for temporarily storing information calculated in the process of various arithmetic operations performed by the control unit 230. As a specific example, the storage unit 220 may store information regarding a target point that is a target of assist of the operation of the arm unit 120, parameters regarding adjustment of a control amount of the assist (hereinafter also referred to as “assist amount”), a point serving as a reference for the control of the operation of the arm unit 120 (hereinafter also referred to as “constraint point”), and the like.

The function and configuration of the control device 20 have been described above. Note that the control device 20 according to the present embodiment can be configured by, for example, various information processing devices (arithmetic processing devices) such as a personal computer (PC) and a server.

The functions and configurations of the arm device 10 and the control device 20 according to the present embodiment have been described above with reference to FIG. 4. Each of the above-described constituent elements may be configured using general-purpose members or circuit, or may be configured by hardware specialized for the function of each constituent element. Furthermore, all the functions of the configuration elements may be performed by a CPU or the like. Therefore, the configuration to be used can be changed as appropriate according to the technical level of the time of carrying out the present embodiment.

### 2.3. Control Example of Medical Arm System

Next, an example of control of the medical arm system according to the present embodiment will be described in more detail.

### 2.3.1. Basic Idea of Arm Control

First, an overview of a basic idea of the technology regarding the arm control based on the setting of the virtual boundary in the medical arm system according to the present embodiment.

In the arm system in related art, for example, a virtual boundary is set in the real space to suppress entry of the distal end unit held by the arm unit into a predetermined region in the real space (for example, in a body). In this case, for example, in a case where the distal end unit comes in contact with the virtual boundary, the position and posture of each joint unit of the arm unit are constrained, and the distal end of the distal end unit is suppressed not to further enter the region separated by the virtual boundary. Meanwhile, in the control using the setting of the virtual boundary in the arm system in related art, for example, a situation to perform an operation to move the distal end unit to a specific position (target point) is not necessarily assumed.

In contrast, in the medical arm system according to the present embodiment, setting of the virtual boundary and the control of the arm unit according to the setting of the virtual boundary are performed to enable assist of the operation to move the point of action (for example, the distal end of the distal end unit) toward the target point.

For example, FIG. 5 is a schematic perspective view for describing an overview of a technology regarding arm control based on setting of the virtual boundary in the medical arm system according to the present embodiment. FIG. 5 schematically illustrates an example of a virtual boundary P10 set in the medical arm system according to the present embodiment. The virtual boundary P10 according to the present embodiment has a surface P11 formed by a flat surface, a curved surface, or a combination thereof, and an opening P13 is set in a part of the surface P11. For example, in the example illustrated in FIG. 5, the virtual boundary P10 has the surface P11 set to be inclined toward the opening P13. More specifically, in the example illustrated in FIG. 5, the virtual boundary P10 has a shape substantially equal to a side surface of a cone with an apex side located downward, and the opening P13 is provided at a position corresponding to the apex side. In other words, the cut section in a case where the virtual boundary P10 is cut in a plane perpendicular to an axis of the cone becomes smaller in area as the virtual boundary P10 is cut at a position closer to the opening P13 (movement target). Note that the dimensions of each part, the shapes of details, and the like of the virtual boundary P10 may be changed as appropriate according to the intended use scene. For example, the virtual boundary P10 may have a shape substantially equal to a side surface of a circular truncated cone with an upper surface side located downward. In this case, the opening P13 (movement target) may be provided at a position corresponding to at least a part of an upper surface (for example, a position corresponding to the upper surface or a position corresponding to a point in the upper surface). Furthermore, FIG. 5 schematically illustrates a distal end portion 141 of the distal end unit 140 held by the arm unit 120. In other words, in the example illustrated in FIG. 5, in a case where the distal end portion 141 comes in contact with the surface P11 of the virtual boundary P10 (in other words, in a case where the distal end portion 141 is located on the surface P11 of the virtual boundary P10), the operation of the arm unit 120 is controlled to suppress entry of the distal end portion 141 into a region on a back surface side separated by the surface P11. Furthermore, at this time, the operation of the arm unit 120 is controlled to assist (support) the movement of the distal end portion 141 in contact with the surface P11 (in other words, the distal end portion 141 located on the surface P11) toward the opening P13 along the surface P11. In other words, it can be said that the opening P13 is set in a part of the surface P11 as the movement target regarding assist of the movement of the distal end portion 141 along the surface P11.

Note that, in the example illustrated in FIG. 5, coordinate axes are defined. Specifically, a direction perpendicular to a center of the opening P13 is defined as the z-axis direction, and directions orthogonal to the z axis and orthogonal to each other are defined as the x-axis direction and the y-axis direction. Furthermore, an up-down direction, a front-back direction, and a right-left direction will be defined in accordance with the coordinate axes for convenience. In other words, the z-axis direction, the x-axis direction, and the y-axis direction are defined as the up-down direction, the right-left direction, and the front-back direction, respectively.

Here, an example of a method of installing the virtual boundary according to the present embodiment will be described with reference to FIG. 6. FIG. 6 is an explanatory diagram for describing an overview of an example of a method of installing the virtual boundary according to the embodiment. Note that the x axis, y axis, and z axis in FIG. 6 correspond to the x axis, y axis, and z axis in FIG. 5, respectively. In FIG. 6, a medical instrument having at least a part inserted into and used in a body of a patient, such as an endoscope, is assumed as the distal end unit 140, for example. In other words, FIG. 6 schematically illustrates a surface M11 of the body of the patient. Furthermore, FIG. 6 schematically illustrates an insertion port M13 used for insertion of the medical instrument into the body of the patient.

Note that, in the present disclosure, the form of the insertion port M13 is not particularly limited as long as the insertion port can be used for the insertion of the medical instrument into the body of the patient. As a specific example, the insertion port M13 may be an insertion port (artificial hole or orifice) formed by installing a so-called trocar or the like. Furthermore, as another example, the insertion port M13 may be an insertion port formed by applying treatment such as incision to the surface M11 of the body. Furthermore, as another example, the insertion port M13 may be an opening (natural hole or orifice) provided as a part of the body, such as an ear canal or a nostril.

In the example illustrated in FIG. 6, the virtual boundary P10 is set in the real space such that the position of the opening P13 of the virtual boundary P10 illustrated in FIG. 5 corresponds to the position of the insertion port M13. Specifically, the position and posture of the virtual boundary P10 are set on the basis of the position of the insertion port M13 such that the distal end portion 141 of the distal end unit 140 (medical instrument) inserted in the opening P13 has a positional relationship of being inserted into the body of the patient via the insertion port M13. Furthermore, the surface P11 of the virtual boundary P10 is set to fall within a predetermined range having the position of the opening P13 as the base point. As a specific example, the surface P11 is set to be inclined toward the opening P13 with the opening P13 as a bottom in a region corresponding to a predetermined range centered on the position of the opening P13 in an xy plane. In other words, in the example illustrated in FIG. 6, the virtual boundary P10 is set to have a so-called mortar shape with an opening provided in the bottom. In other words, in the virtual boundary P10, the opening P13 is set such that the position corresponding to the insertion port M13 in the surface P11 becomes insertable.

With the above configuration, for example, proximity of the distal end portion 141 to a portion other than the opening P13 of the surface M11 of the body of the patient is blocked by the surface P11 of the virtual boundary P10. Therefore, occurrence of a situation where the distal end portion 141 comes in contact with the surface M11 can be prevented. Furthermore, the movement of the distal end portion 141 (point of action) in contact with the surface P11 toward the opening P13 (movement target) along the surface P11 is assisted (supported). Therefore, the operation to insert the distal end portion 141 into the insertion port M13 can be assisted. In other words, the movement of the arm unit 120 is controlled such that the movable range of the point of action (for example, the distal end unit 141) is further restricted as the point of action gets closer to the target point (for example, the insertion port M13), for example, on the basis of the setting of the virtual boundary P10 according to the present embodiment.

An overview of the basic idea of the technology regarding the arm control based on the setting of the virtual boundary in the medical arm system according to the present embodiment has been described with reference to FIGS. 5 and 6.

### 2.3.2. Comparative Example: Operation Suppression Control

Next, to make the characteristics of the arm control by the medical arm system according to the present embodiment more understandable, an example of arm control for the purpose of suppression of entry of the distal end unit to a predetermined region in the real space will be described as a comparative example.

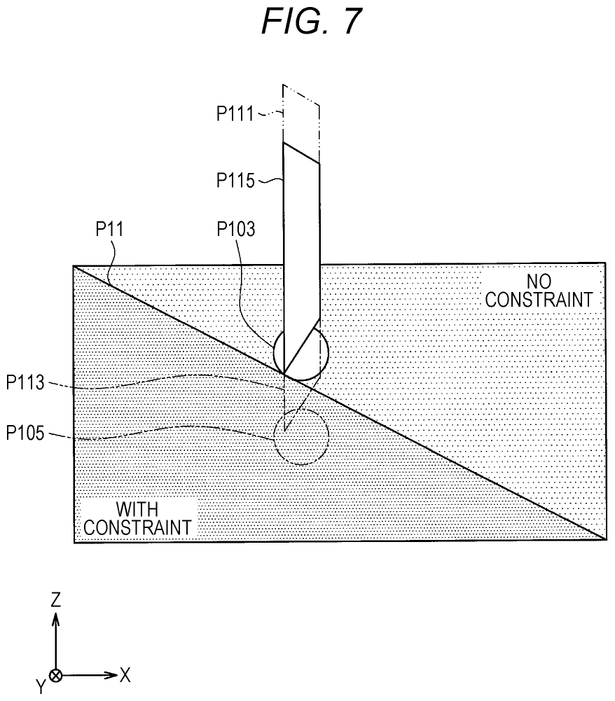

First, an overview of arm control according to a comparative example will be described with reference to FIG. 7. FIG. 7 is an explanatory diagram for describing an overview of an example of arm control in an arm system according to a comparative example. In the example illustrated in FIG. 7, a use case is assumed in which an endoscope is applied as the distal end unit 140, and the endoscope is inserted into an insertion port formed using a trocar or the like. Furthermore, the x axis, y axis, and z axis in FIG. 7 correspond to the x axis, y axis, and z axis in FIG. 5, respectively.

FIG. 7 illustrates a surface P11 (hereinafter also referred to as a “boundary surface”) of a virtual boundary set in the real space. In other words, the boundary surface P11 corresponds to the surface P11 of the virtual boundary P10 illustrated in FIGS. 5 and 6. Furthermore, FIG. 7 schematically illustrates positions P111, P113, and P115 of the distal end unit 140 in the process of an operation to move the distal end unit 140 from above the boundary surface P11 toward the boundary surface P11 (in other words, downward). Specifically, the position P111 represents the position of the distal end unit 140 before the distal end portion 141 of the distal end unit 140 comes in contact with the boundary surface P11. Furthermore, the position P113 represents, as a result of the above operation, the position of the distal end unit 140 in a case where the distal end portion 141 has entered a region separated by the boundary surface P11 (in other words, a region below the boundary surface P11) under a situation where the distal end portion 141 of the distal end unit 140 is predicted to enter the region. Note that FIG. 7 schematically illustrates a position P105 of the distal end portion 141 at that time. Furthermore, the position P115 represents the position of the distal end unit 140 in a case where the operation of the arm unit 120 is controlled to suppress entry of the distal end portion 141 into the region separated by the boundary surface P11.

In the example illustrated in FIG. 7, in a case where the distal end portion 141 (point of action) of the distal end unit 140 is located in a region above the boundary surface P11, the movement of the distal end unit 140 (in other words, the operation of the arm unit 120) is not constrained. In contrast, in a case where entry of the distal end portion 141 into a region below the boundary surface P11 is predicted (or in a case where entry of the distal end portion 141 into the region has occurred), the movement of the distal end unit 140 (in other words, the operation of the arm unit 120) is constrained to suppress the entry of the distal end portion 141 into the region. Specifically, a constraint point P103 is set on the boundary surface P11 where the boundary surface P11 and the distal end portion 141 are in contact, and a constraint condition of translational three degrees of freedom in xyz directions according to the position of the constraint point P103 is given to the condition of the operation control of the arm unit 120. Thereby, the movement of the distal end unit 140 (in other words, the operation of the arm unit 120) is suppressed such that the distal end portion 141 is located on the boundary surface P11. At this time, movement of the distal end unit 140 other than the movement toward the region above the boundary surface P11 where the movement of the distal end unit 140 is not constrained is restricted.

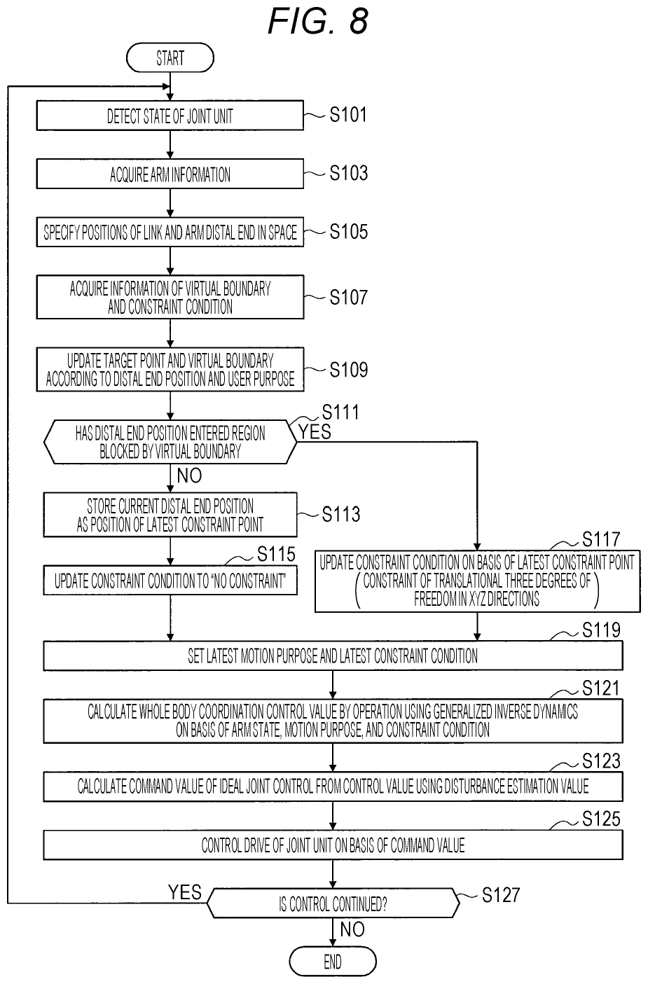

Here, an example of a flow of a series of processing of the arm system according to the comparative example will be described with reference to FIG. 8, especially focusing on the control of the movement of the distal end unit 140 (in other words, control of the movement of the arm unit 120) according to the setting of the virtual boundary. FIG. 8 is a flowchart illustrating an example of a flow of a series of processing of the arm system according to the comparative example.

As illustrated in FIG. 8, the arm device 10 (joint state detection unit 132) detects the state of the joint units 130 configuring the arm unit 120 (S101) and transmits the detection result to the control device 20 as the arm information. The control device 20 (arm state acquisition unit 240) acquires the arm information according to the state of the arm from the arm device 10 (S103) and specifies the positions (coordinates) in the space of the links and distal end unit 140 and the force acting on the joint units 130, the links, and the distal end unit 140, and the like on the basis of the arm information (S105).

Next, the control device 20 (virtual boundary update unit 251 and constraint condition update unit 255) acquires the information regarding the virtual boundary and the information regarding the constraint condition related to the control of the operation of the arm unit 120 (for example, the information of the latest constraint condition) (S107). The control device 20 (virtual boundary update unit 251) sets and updates the virtual boundary on the basis of various conditions. For example, the control device 20 may set and update the target point according to the position of the distal end portion 141 of the distal end unit 140 (the position of the point of action) and the instruction (user purpose) by the user, and set and update the virtual boundary according to the setting of the target point (S109).

The control device 20 (region entry determination unit 253) determines entry of the distal end portion 141 of the distal end unit 140 (point of action) into the region separated by the virtual boundary on the basis of the result of the setting and update of the virtual boundary and the arm information (S111). In a case where it is determined that the distal end portion 141 has not entered the region (S111, NO), the control device 20 (constraint condition update unit 255) stores the current position of the distal end portion 141 as the latest position of the constraint point (S113) and updates the constraint condition with no constraint (S115). In other words, in this case, the operation of the arm unit 120 is not suppressed.

Meanwhile, in a case where it is determined that the distal end portion 141 has entered the region (S111, YES), the control device 20 (region entry determination unit 253) updates the constraint condition to suppress at least a part of the operation of the arm unit 120 to suppress the entry of the distal end portion 141 into the region on the basis of the latest constraint point. As a specific example, the control device 20 may update the constraint condition such that the distal end portion 141 is located on the surface of the virtual boundary by constraining the translational three degrees of freedom in the xyz directions (S117), as described with reference to FIG. 7. Furthermore, the control device 20 (motion purpose update unit 257) may update the motion condition regarding the control of the operation of the arm unit 120 in response to the update of the constraint condition.

Next, the control device 20 (arithmetic condition setting unit 260) sets the latest motion purpose and the latest constraint condition as the arithmetic operation condition in the arithmetic operation regarding the whole body coordination control using the generalized inverse dynamics in order to realize a manual operation using an external force as an operation force (S119).

The control device 20 (whole body coordination control unit 270) calculates the control command value for the whole body coordination control by the arithmetic operation using the generalized inverse dynamics on the basis of the state of the arm, the motion purpose, and the constraint condition (S121). Notably, whilst the whole body coordination control unit 270 of the control device has been described herein as calculating the control command value for the whole body coordination control, for example using inverse dynamics, this is a non-limiting example. Rather, any suitable technique for control of some or all of the multilink structure (or any other form of articulated medical arm) may be considered.

The control device 20 (ideal joint control unit 280) calculates the disturbance estimation value τ_{d} on the basis of the torque command value τ and the rotation angular speed calculated from the rotation angle q of the joint unit 130 configuring the arm unit 120. Furthermore, the control device 20 calculates the torque command value τ that is a command value representing the torque to be generated in the arm unit 120 and finally transmitted to the arm device 10, using the disturbance estimation value τ_{d} (S123).

As described above, the control device 20 transmits the calculated torque command value τ to the arm device 10. Then, the arm device 10 (drive control unit 111) performs control to supply the current amount corresponding to the torque command value τ transmitted from the control device 20 to the motor in the actuator of the joint unit 130, thereby controlling the number of rotations of the motor and controlling the rotation angle and the generated torque in the joint unit 130 (S125).

A series of processing as described above is sequentially executed as long as the control continues (S127, YES). Then, when termination of control is given in instruction by power OFF or the like (S127, NO), execution of the above-described series of processing is terminated.

An example of the arm control mainly for the suppression of entry of the distal end unit into a predetermined region in the real space has been described with reference to FIGS. 7 and 8, as a comparative example.

Meanwhile, under a situation where the arm control according to the above-described comparative example is performed, there are cases where a complicated operation is necessary (in other words, the operability is reduced) to realize the user operation to move the distal end portion 141 to a specific position, for example. Specifically, under the above-described control, the user performs the operation while confirming the shape of the virtual boundary in an exploratory manner, or performs the operation while confirming the shape of the virtual boundary using a display device or the like, for example. In view of such a situation, in control examples to be described below, setting of the virtual boundary and the control of the arm unit 120 according to the setting of the virtual boundary are performed to enable assist of the operation to move the point of action (for example, the distal end of the distal end unit) toward the target point, thereby improving the operability. Therefore, hereinafter, examples of the arm control according to an embodiment of the present disclosure will be described as a first control example and a second control example.

### 2.3.3. First Control Example: Operation Assist Control by Position Update of Constraint Point

First, as the first control example, an example of control for assisting (supporting) the user operation by updating the position of the constraint point according to the positional relationship between the virtual boundary and the point of action will be described.

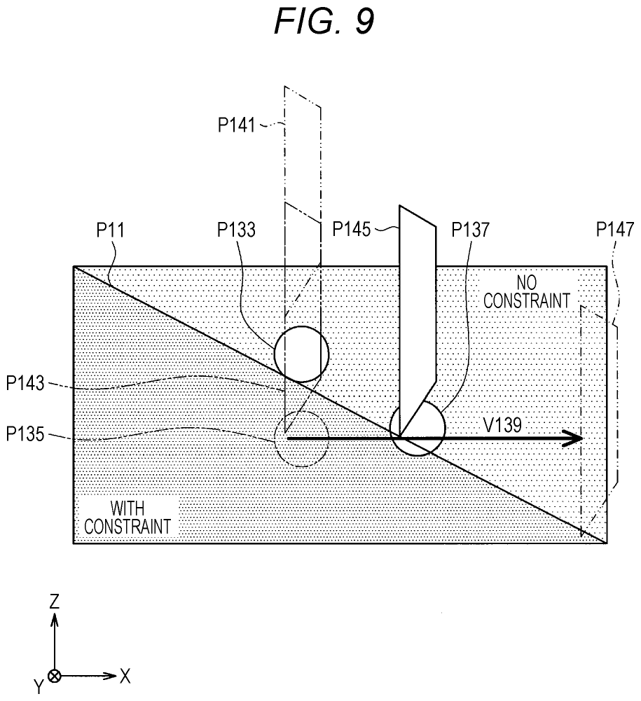

First, an overview of the arm control according to the first control example will be described with reference to FIG. 9. FIG. 9 is an explanatory diagram for describing an overview of the arm control according to the first control example, illustrating an example of the arm control in the medical arm system according to an embodiment of the present disclosure. In the example illustrated in FIG. 9, a use case is assumed in which an endoscope is applied as the distal end unit 140, and the endoscope is inserted into an insertion port formed using a trocar or the like. Furthermore, the x axis, y axis, and z axis in FIG. 9 correspond to the x axis, y axis, and z axis in FIG. 5, respectively.

FIG. 9 illustrates a surface P11 (in other words, boundary surface) of a virtual boundary set in the real space, which corresponds to the surface P11 of the virtual boundary P10 illustrated in FIGS. 5 and 6. Furthermore, FIG. 9 schematically illustrates positions P141, P143, and P145 of the distal end unit 140 in the process of an operation to move the distal end unit 140 from above the boundary surface P11 toward the boundary surface P11 (in other words, downward). Specifically, the position P141 represents the position of the distal end unit 140 before the distal end portion 141 of the distal end unit 140 comes in contact with the boundary surface P11. Furthermore, the position P143 represents, as a result of the above operation, the position of the distal end unit 140 in a case where the distal end portion 141 has entered a region separated by the boundary surface P11 (in other words, a region on a lower side of the boundary surface P11) under a situation where the distal end portion 141 of the distal end unit 140 is predicted to enter the region. Note that FIG. 9 schematically illustrates a position P135 of the distal end portion 141 at that time. Furthermore, the position P145 represents the position of the distal end unit 140 in a case where the operation of the arm unit 120 is controlled to suppress entry of the distal end portion 141 into the region separated by the boundary surface P11.

In the example illustrated in FIG. 9, in the case where the distal end portion 141 (point of action) of the distal end unit 140 is located in a region above the boundary surface P11, the movement of the distal end unit 140 (in other words, the operation of the arm unit 120) is not constrained. Note that, in the following description, the region is also referred to as a “non-constraint condition region” for convenience.

In contrast, in a case where entry of the distal end portion 141 into a region below the boundary surface P11 is predicted (or in a case where entry of the distal end portion 141 into the region has occurred), the entry of the distal end portion 141 into the region is suppressed, and the movement of the distal end portion 141 along the boundary surface P11 toward the position set as the movement target is assisted. Note that the example in FIG. 9 schematically illustrates a position of the movement target P147. Furthermore, in the following description, the region where the movement of the distal end unit 140, like the region below the boundary surface P11 in the example in FIG. 9, is constrained is also referred to as “constraint condition region” for convenience.

Specifically, the position on the boundary surface P11 at which the distal end portion 141 enters the constraint condition region (hereinafter, also referred to as “entry point P133”) and an entry direction into the region are calculated on the basis of the detection result of the contact between the boundary surface P11 and the distal end portion 141 (in other words, the detection result of the distal end portion 141 being located on the boundary surface P11). Next, a position different from the entry point P133 present in the non-constraint condition region is set as a latest constraint point P137 on the basis of the shape of the boundary surface P11 and the movement target P147. For example, in the example illustrated in FIG. 9, the constraint point P137 is set on the position on the boundary surface P11 at which the boundary surface P11 intersects with a vector V139 from the position P135 of the distal end portion 141 toward the movement target P147 in the case where the distal end portion 141 has entered the constraint condition region as a result of the operation. After the setting of the latest constraint point P137, the constraint condition of translational three degrees of freedom in xyz directions according to the position of the constraint point P137 is given to the condition of the operation control of the arm unit 120. As a result, the constraint condition is updated to prompt the movement of the distal end portion 141 toward the movement target P147. In other words, the movement of the distal end unit 140 (in other words, the operation of the arm unit 120) is controlled such that the distal end portion 141 is located on the boundary surface P11 and the movement of the distal end portion 141 along the boundary surface P11 toward the movement target P147 is assisted. As discussed later herein with reference to a summary embodiment, this assistance capability may be used to provide guidance for a user, for example to indicate a preferred route by selective exertion of reactive and/or resistive forces that prevent and/or resist motion outside a preferred path of approach to the target. Hence the control unit may be adapted to apply a generated force in the articulated medical arm system in response to a guidance rule. In some examples of the guidance rule, it may be a rule defining what force is applied on the medical arm to guide, for example, the distal end portion 141 (referred to as a predetermined point/a point of action) of the distal end unit 140 to the movement target P147. For example, it may include a rule for generating impetus (pushing and/or pulling force) to assist a movement of the distal end portion 141 towards the movement target P147, and a rule for generating a reaction force or a resistance force to a movement of the distal end portion 141 to a direction not towards the movement target P147. In addition to these rules, the other guidance rule may include a rule to add offset (it may increase by steps) to the impetus, the reaction force and/or the resistive force and a rule to increase these forces (by steps), to realize a more careful movement of the distal end portion 141 where it comes close to the movement target P147.

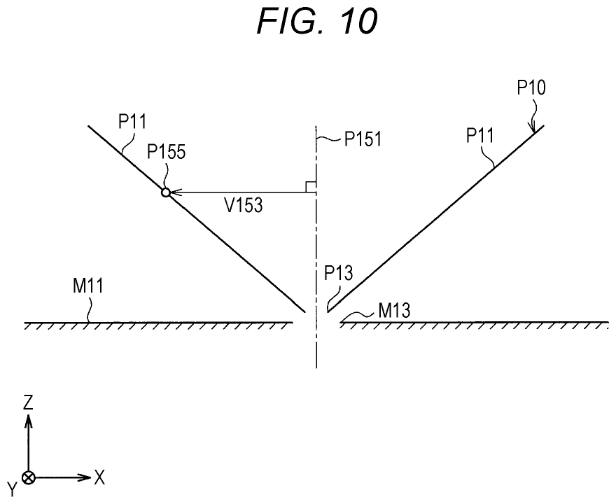

Furthermore, FIG. 10 is an explanatory diagram for describing an example of a method of setting the constraint point in the arm control according to the first control example. In other words, FIG. 10 illustrates an example of a method of setting a position (hereinafter also referred to as “entry suppression point”) on the boundary surface P11 at which entry of the point of action into the non-constraint condition region separated by the boundary surface P11 of the virtual boundary P10 is suppressed. In FIG. 10, similar reference numerals to FIG. 5 similarly represent objects denoted with the reference numerals in the example illustrated in FIG. 5. Furthermore, FIG. 10 illustrates an entry suppression point (constraint point) P155 set on the boundary surface P11 of the virtual boundary P10. Furthermore, an axis P151 is perpendicular to the center of the opening P13 (in other words, the center of the insertion port M13). In other words, the axis P151 corresponds to an axis set to be inserted into both the opening P13 and the insertion port M13. Furthermore, a vector V153 perpendicularly intersects with the axis P151. In other words, the entry suppression point P155 can be set on the boundary surface P11 by using a calculation result of an intersection of the vector V153 perpendicular to the axis P151 and the boundary surface P11 of the virtual boundary P10.

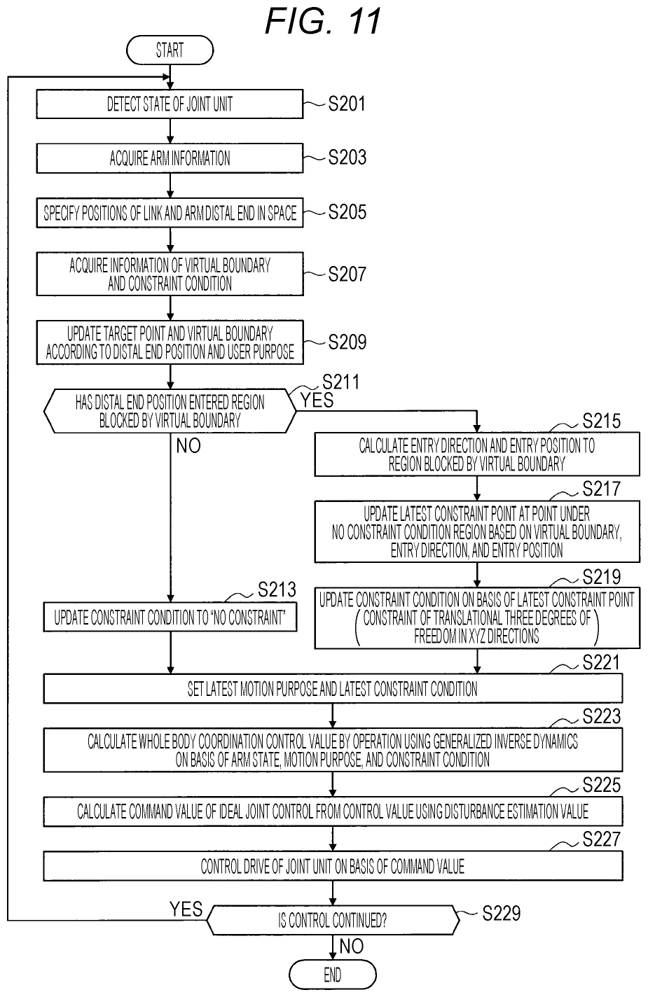

Next, an example of a flow of a series of processing of the arm control according to the first control example will be described with reference to FIG. 11, especially focusing on the control of the movement of the distal end unit 140 (in other words, control of the movement of the arm unit 120) according to the setting of the virtual boundary. FIG. 11 is a flowchart illustrating an example of a flow of a series of processing of the arm control according to the first control example. Note that the processing represented in reference numerals S201 to S209 are substantially similar to the processing represented by reference numerals S101 to S109 in the example illustrated in FIG. 8, and therefore detailed description is omitted.

The control device 20 (region entry determination unit 253) determines entry of the distal end portion 141 of the distal end unit 140 (point of action) into the region (non-constraint condition region) separated by the virtual boundary on the basis of the result of the setting and update of the virtual boundary and the arm information (S211). In a case where it is determined that the distal end portion 141 has not entered the constraint condition region (S211, NO), the control device 20 (constraint condition update unit 255) updates the constraint condition with no constraint (S213). In other words, in this case, the operation of the arm unit 120 is not suppressed.

On the other hand, in a case where it is determined that the distal end portion 141 has entered the constraint condition region (S211, YES), the control device 20 (region entry determination unit 253) calculates the entry direction and the entry position of the distal end portion 141 into the constraint condition region (S215). Note that the entry direction and the entry position of the distal end portion 141 (point of action) into the constraint condition region can be calculated according to the relative relationship between the position of the distal end unit 140 according to the state of the arm unit 120 and the position of the virtual boundary P10.

Next, the control device 20 (constraint condition update unit 255) updates the constraint point so that the position different from the entry position present in the non-constraint condition region becomes the latest constraint point on the basis of the virtual boundary and the calculation result of the entry direction and the entry position (S217). Then, the control device 20 (region entry determination unit 253) updates the constraint condition to suppress at least a part of the operation of the arm unit 120 on the basis of the latest constraint point. As a specific example, the control device 20 may update the constraint condition to suppress the entry of the distal end portion 141 (point of action) into the constraint condition region by constraining the translational three degrees of freedom in the xyz directions and to assist the movement of the distal end portion 141 along the boundary surface of the virtual boundary toward the movement target (S219), as described with reference to FIG. 9. Furthermore, the control device 20 (motion purpose update unit 257) may update the motion condition regarding the control of the operation of the arm unit 120 in response to the update of the constraint condition.

Note that subsequent operations (in other words, reference numerals S221 to S229) are substantially similar to the example described with reference to FIG. 8, and therefore detailed description is omitted.

By the above control, assist of a moving operation along the boundary surface of the virtual boundary according to an operation target of the user becomes possible in addition to suppression of the entry of the point of action (for example, the distal end portion 141) into the region separated by the virtual boundary. As a specific example, under a situation where an endoscope is inserted into an insertion port formed by using a trocar or the like, the distal end can be guided along the boundary surface by pushing the distal end of the endoscope against the boundary surface of the virtual boundary. In other words, it is possible to assist and/or guide the user's operation to move the endoscope toward the insertion port without causing the user to be conscious of the operation toward the insertion port as the target position. As described herein, the control device can suppress unwanted movement through the virtual boundary, and can also optionally apply a force to push the point of action on to the boundary, and/or towards the operation target. Furthermore, the shape of the virtual boundary and the position of the opening (in other words, the position of the operation target) can be appropriately set or updated according to various conditions. Therefore, for example, by setting or updating the operation target and the shape of the virtual boundary by combining the above control with a position memory function to store a position during operation, it is also possible to assist the user's operation to move the distal end unit held by the arm toward a specific memory position.

As described above, as the first control example, the example of control for assisting (supporting) the user operation by updating the position of the constraint point according to the positional relationship between the virtual boundary and the point of action has been described with reference to FIGS. 9 to 11.

### 2.3.4. Second Control Example: Operation Assist Control by Force Control

Next, as the second control example, an example of control for assisting (supporting) the user operation by estimating an external force applied from the point of action to the virtual boundary and simulating a reaction force against the external force will be described.

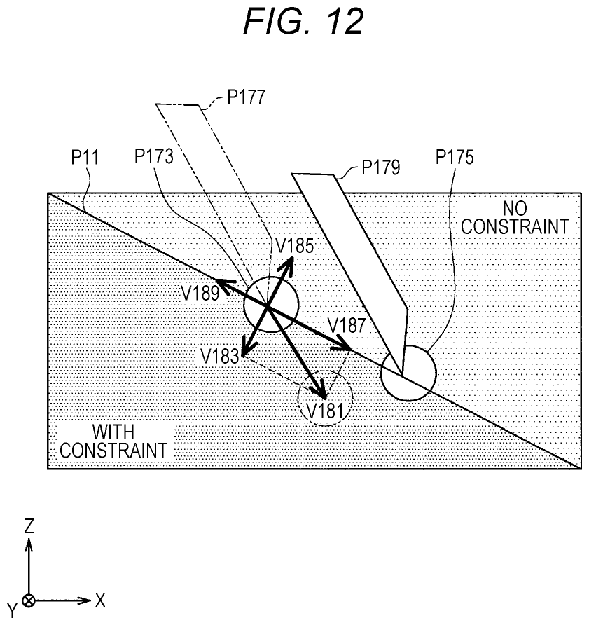

First, an overview of arm control according to the second control example will be described with reference to FIG. 12. FIG. 12 is an explanatory diagram for describing an overview of the arm control according to the second control example, illustrating an example of the arm control in the medical arm system according to an embodiment of the present disclosure. In the example illustrated in FIG. 12, a use case is assumed in which an endoscope is applied as the distal end unit 140, and the endoscope is inserted into an insertion port formed using a trocar or the like. Furthermore, the x axis, y axis, and z axis in FIG. 12 correspond to the x axis, y axis, and z axis in FIG. 5, respectively.

FIG. 12 illustrates a surface P11 (in other words, boundary surface) of a virtual boundary set in the real space, which corresponds to the surface P11 of the virtual boundary P10 illustrated in FIGS. 5 and 6. Furthermore, FIG. 12 schematically illustrates positions P177 and P179 of the distal end unit 140 in the process of an operation to press the distal end unit 140 against the boundary surface P11. Specifically, the position P177 represents the position of the distal end unit 140 at timing when the operation to press the distal end unit 140 against the boundary surface P11 has been performed in a state where the distal end portion 141 of the distal end unit 140 is in contact with the boundary surface P11 (in other words, a state where the distal end portion 141 is located on the boundary surface P11). Furthermore, the position P179 represents the position of the distal end unit 140 (the position of the distal end unit 140 after operation) in a case where the operation of the arm unit 120 is controlled to suppress entry of the distal end portion 141 into the region separated by the boundary surface P11, in response to the above operation.

In the example illustrated in FIG. 12, in the case where the distal end portion 141 (point of action) of the distal end unit 140 is located in a region above the boundary surface P11, the movement of the distal end unit 140 (in other words, the operation of the arm unit 120) is not constrained. This point is similar to the first control example described with reference to FIG. 9.

When the operation to further move the distal end portion 141 in contact with the boundary surface P11 toward the region separated by the boundary surface P11 is performed, a reaction force to suppress the entry of the distal end portion 141 into the region is simulated. Specifically, in a case where it is assumed that the boundary surface P11 is actually present as an object, the external force acting from the distal end portion 141 in contact with the boundary surface P11 (in other words, the distal end portion 141 located on the boundary surface P11) on the boundary surface P11 is estimated. For example, FIG. 12 schematically illustrates a position P173 at which the distal end portion 141 of the distal end unit 140 located at the position P177 is in contact with the boundary surface P11. Furthermore, a vector V181 represents a vector of the external force estimated to be applied from the distal end unit 140 located at the position P177 to the boundary surface P11. Furthermore, a vector V183 represents a vector of a vertical component with respect to the boundary surface P11, of the vector V181 of the external force. Furthermore, a vector V187 represents a vector of a horizontal component with respect to the boundary surface P11, of the vector V181 of the external force.

Furthermore, the vector V183 of the vertical component with respect to the boundary surface P11 is calculated from the estimation result of the external force illustrated as the vector V181, so that a vector V185 of the reaction force that cancels the influence of the vertical component can be calculated. In other words, in the example illustrated in FIG. 12, the operation of the arm unit 120 is controlled such that the reaction force in the vertical direction illustrated as the vector V185 is simulated, so that the entry of the distal end portion 141 into the region (constraint condition region) below the boundary surface P11 can be suppressed. Furthermore, the horizontal component illustrated as the vector V183 remains without being canceled, so that the movement of the distal end unit 140 (in other words, the operation of the arm unit 120) is controlled such that the movement of the distal end portion 141 along the boundary surface P11 is assisted. Note that the reaction force in the vertical direction illustrated as the vector V185 corresponds to an example of “first reaction force”.

Note that the horizontal component of the external force with respect to the boundary surface P11 becomes able to be calculated, as illustrated as the vector V187. Therefore, for example, a vector V189 of the reaction force to restrict (cancel, accordingly) the influence of the horizontal component can be calculated. Therefore, for example, the operation of the arm unit 120 is controlled such that the reaction force in the horizontal direction illustrated as the vector V189 is simulated, so that an assist amount regarding the movement of the distal end portion 141 along the boundary surface P11 can be adjusted. Note that the reaction force in the horizontal direction illustrated as the vector V189 corresponds to an example of “first reaction force”.

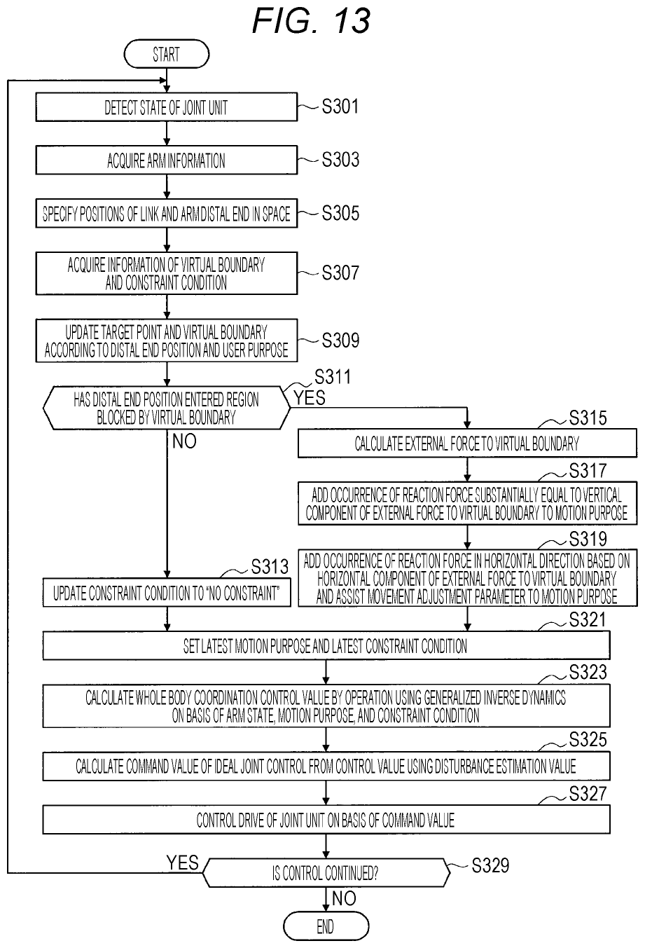

Next, an example of a flow of a series of processing of the arm control according to the second control example will be described with reference to FIG. 13, especially focusing on the control of the movement of the distal end unit 140 (in other words, control of the movement of the arm unit 120) according to the setting of the virtual boundary. FIG. 13 is a flowchart illustrating an example of a flow of a series of processing of the arm control according to the second control example. Note that the processing represented in reference numerals S301 to S309 are substantially similar to the processing represented by reference numerals S101 to S109 in the example illustrated in FIG. 8, and therefore detailed description is omitted.

The control device 20 (region entry determination unit 253) determines entry of the distal end portion 141 of the distal end unit 140 (point of action) into the region (non-constraint condition region) separated by the virtual boundary on the basis of the result of the setting and update of the virtual boundary and the arm information (S311). In a case where it is determined that the distal end portion 141 has not entered the constraint condition region (S311, NO), the control device 20 (constraint condition update unit 255) updates the constraint condition with no constraint (S313). In other words, in this case, the operation of the arm unit 120 is not suppressed.

On the other words, in a case where it is determined that the distal end portion 141 has entered the constraint condition region (S211, YES), the control device 20 (region entry determination unit 253) calculates (estimates) the external force acting on the boundary surface P11 from the distal end portion 141 (point of action) in contact with the boundary surface P11 of the virtual boundary P10. Note that the vector of the external force acting from the distal end portion 141 (point of action) on the boundary surface P11 can be calculated according to the relative relationship between the position of the distal end unit 140 according to the state of the arm unit 120 and the position of the virtual boundary P10, and the shape of the virtual boundary P10 and the like.

Next, the control device 20 (constraint condition update unit 255 and motion purpose update unit 257) calculates the vector of the vertical component of the external force with respect to the boundary surface P11 on the basis of the calculation result of the external force acting on the boundary surface P11 of the virtual boundary P10. Then, the control device 20 calculates the vector of the reaction force that cancels the influence of the vertical component on the basis of the calculation result of the vector of the vertical component. In other words, the control device 20 updates the constraint condition and the motion purpose such that the reaction force against a vertical component approximately equal in magnitude to the vertical component of the external force with respect to the boundary surface P11 is generated (S317).

Furthermore, the control device 20 (constraint condition update unit 255 and motion purpose update unit 257) may adjust the assist amount regarding the movement of the distal end portion 141 along the boundary surface P11 by calculating the vector of the horizontal component of the external force with respect to the boundary surface P11 of the virtual boundary P10. Specifically, the control device 20 calculates the vector of the reaction force that restricts the influence of the horizontal component on the basis of the calculation result of the vector of the horizontal component of the external force. At this time, the control device 20 may control a restriction amount of the influence of the horizontal component (in other words, the magnitude of the reaction force against the horizontal component) according to an adjustment parameter regarding the assist amount regarding the movement of the distal end portion 141 (point of action). As described above, the control device 20 updates the constraint condition and the motion purpose such that the reaction force against the horizontal component according to the magnitude of the horizontal component of the external force with respect to the boundary surface P11 (S319).

Note that subsequent operations (in other words, reference numerals S321 to S329) are substantially similar to the example described with reference to FIG. 8, and therefore detailed description is omitted.

By the above control, assist of the moving operation along the boundary surface of the virtual boundary according to the force applied to the arm unit by the operation of the user (in other words, the external force to move the point of action) becomes possible in addition to suppression of the entry of the point of action (for example, the distal end portion 141) into the region separated by the virtual boundary. Furthermore, at this time, a reaction force according to the horizontal component of the boundary surface, of the estimation result of the external force with respect to the boundary surface of the virtual boundary from the point of action based on the operation from the user, can be generated. By generating such a reaction force, for example, control of a moving amount of generating resistance becomes possible against the moving operation along the boundary surface of the virtual boundary according to the force applied to the arm unit by the operation of the user, for example. In other words, by generating a reaction force according to the horizontal component of the boundary surface, a frictional force against the operation of the user toward the operation target can be simulated.

As the second control example, the example of control for assisting (supporting) the user operation by estimating an external force applied from the point of action to the virtual boundary and simulating a reaction force against the external force has been described with reference to FIGS. 12 and 13.

### 2.3.5. First Example: Operation Assist Control Example Using Virtual Boundary

Next, as the first example, as an example of control regarding assist of the user operation using the virtual boundary by the system according to an embodiment of the present disclosure, an example of the arm control based on the setting of the virtual boundary assuming a situation of assisting insertion of a distal end of an endoscope into a port will be described.

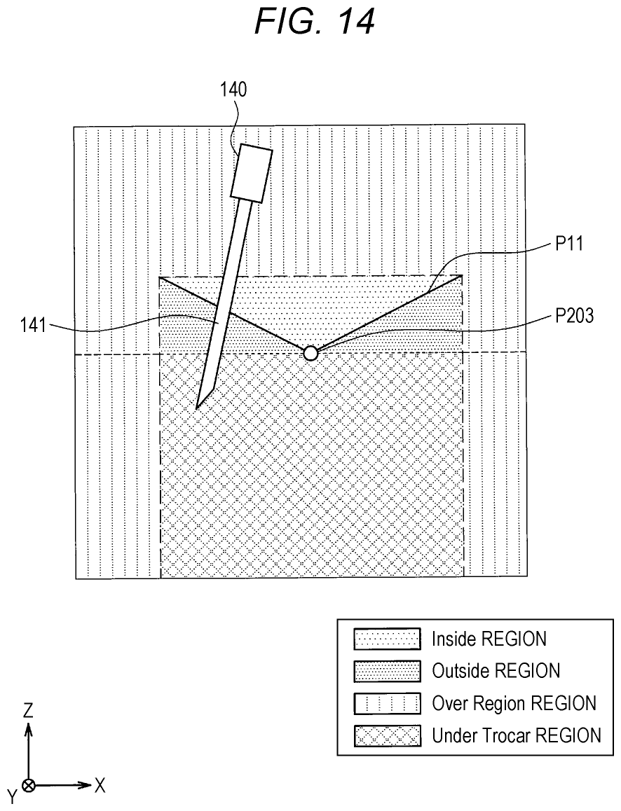

First, an overview of the arm control according to the first example will be described with reference to FIG. 14. FIG. 14 is an explanatory diagram for describing an overview of the arm control according to the first example. In the example illustrated in FIG. 14, an endoscope is applied as the distal end unit 140, and the boundary surface P11 of the virtual boundary is set to assist introduction of the distal end portion 141 (in other words, a distal end of a lens barrel) of the endoscope into an insertion port P203 for inserting a medical instrument into a body, the insertion port being provided by installation of a trocar or the like. In other words, in the example illustrated in FIG. 14, an opening of the virtual boundary is located at a position corresponding to the insertion port P203, and the boundary surface P11 of the virtual boundary is set to be inclined toward the opening. Note that the setting condition of the opening is not particularly limited. As a specific example, in the case of using the trocar, the shape of the virtual boundary may be set or updated as appropriate according to the posture of the trocar and the direction of the insertion port of the trocar.

Furthermore, in the example illustrated in FIG. 14, “Inside region”, “Outside region”, “Over Region region”, and “Under Trocar region” are set in accordance with the setting of the virtual boundary. The Inside region corresponds to a region opposite to a region where the body of the patient is located, of two regions separated by the boundary surface P11, and corresponds to a region above the boundary surface P11 in the example illustrated in FIG. 14. In contrast, the Outside region corresponds to the region opposite to the Inside region, of the two regions separated by the boundary surface P11, and corresponds to a region below the boundary surface P11 in the example illustrated in FIG. 14. The Under Trocar region corresponds to a region into which the distal end portion 141 (point of action) of the distal end unit 140 is introduced through the insertion port P203, and corresponds to, for example, a region corresponding to the inside of the body of the patient. Furthermore, the Over Region region schematically indicates a region to which the condition regarding the arm control is not applied.

Each of the Inside region and the Over Region region corresponds to the region (non-constraint condition region) where the movement of the distal end unit 140 is not constrained. In contrast, each of the Outside region and the Under Trocar region corresponds to the region (constraint condition region) where the movement of the distal end unit 140 is constrained. The range of the constraint condition region is restricted according to the setting of the virtual boundary in this way, so that the target range of the arm control can be set to the minimum necessary, and a free operation without constraint can be realized outside the range without depending on the position or posture of the distal end unit 140.

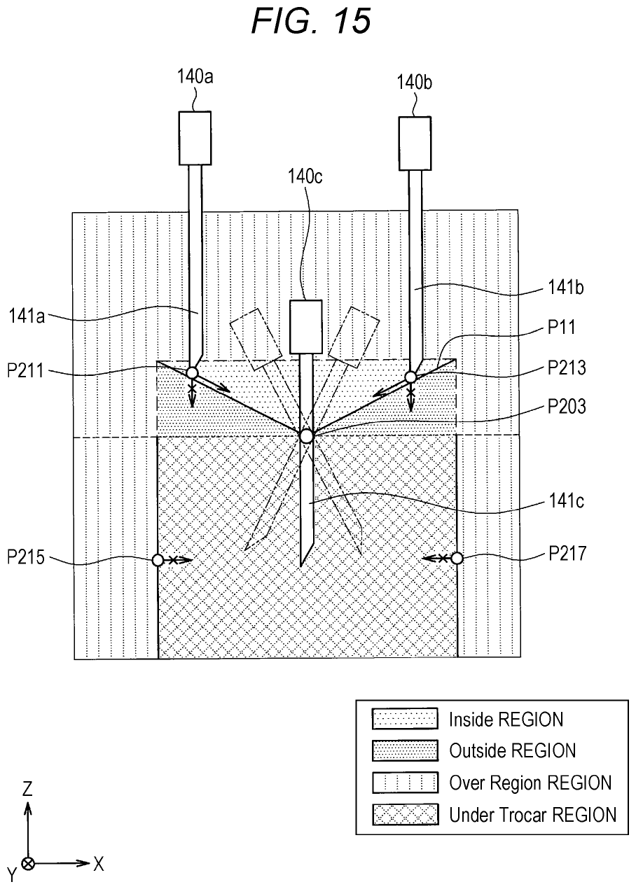

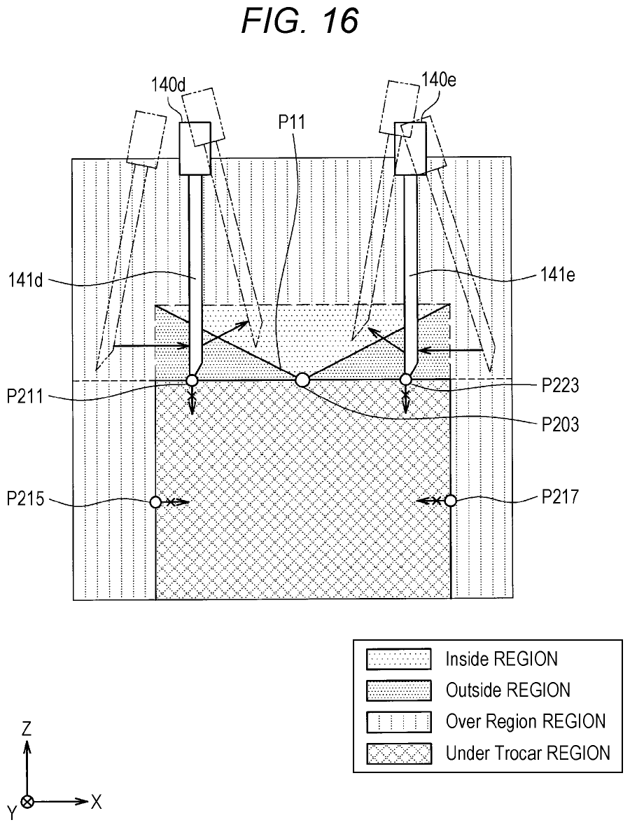

Here, a specific example of the arm control will be described with reference to FIGS. 15 and 16. Each of FIGS. 15 and 16 is an explanatory diagram for describing an overview of an example of arm control according to the first example.

First, an example of the arm control according to the first example will be described with reference to FIG. 15. FIG. 15 schematically illustrates positions and postures 140a to 140c of the distal end unit 140. Furthermore, FIG. 15 illustrates distal end portions 141a to 141c of the distal end units 140a to 140c, respectively.

In the Outside region, entry (transition) of the distal end unit 140 from the Inside region is suppressed according to the setting of the boundary surface P11. As a specific example, in the example illustrated in FIG. 15, the distal end portion 141a of the distal end unit 140a is in contact with the boundary surface P11 from the Inside region side, at another position P211 other than the position of the boundary surface P11 where the opening is set (in other words, the position corresponding to the insertion port P203) In this case, entry of the distal end portion 141a from the position P211 into the Outside region is suppressed by the arm control. Meanwhile, the movement of the distal end portion 141a along the boundary surface P11 is not constrained. Therefore, as illustrated in FIG. 15, when the arm is operated to press the distal end portion 141a against the position P211 on the boundary surface P11, the movement of the distal end portion 141a along the inclination of the boundary surface P11 toward the insertion port P203 (in other words, the opening of the virtual boundary) is assisted. The distal end portion 141b of the distal end unit 140b in contact with the boundary surface P11 is similarly assisted at a position P213 on the boundary surface P11 from the Inside region side.

In the Under Trocar region, entry (transition) from the Inside region through the insertion port P203 (in other words, the opening of the virtual boundary) is permitted, and entry (transition) from other parts is suppressed. For example, in the example illustrated in FIG. 15, the distal end portion 141c of the distal end unit 140c is inserted into the insertion port P203, thereby entering the Under Trocar region from the Inside region. In the state where the distal end portion 141c has entered the Under Trocar region through the insertion port P203, as described above, at least a part of the movement of the distal end unit 140c may be constrained. As a specific example, the distal end unit 140c may be constrained in translational two degrees of freedom in the XY directions. In other words, the distal end unit 140c may be permitted to move only in the Z direction. Furthermore, in the example illustrated in FIG. 15, there is a portion where the Over Region region and the Under Trocar region are in contact as in positions P215 and P217. Even in such a case, entry into the Under Trocar region from the Over Region region is suppressed.

Next, another example of the arm control according to the first example will be described with reference to FIG. 16. FIG. 16 schematically illustrates positions and postures 140d to 140e of the distal end unit 140. Furthermore, FIG. 16 illustrates distal end portions 141d to 141e of the distal end units 140d to 140e, respectively.

As described above, the entry (transition) of the point of action (for example, the distal end unit 140) into the Outside region from the Inside region separated by the boundary surface P11 is suppressed. Meanwhile, the entry (transition) of the point of action from the Outside region into the Inside region may be permitted. As a specific example, in the example illustrated in FIG. 16, the distal end portion 141d of the distal end unit 140d is located in the Outside region. Under such a situation, in a case where an operation to cause the distal end portion 141d to enter the Inside region beyond the boundary surface P11 from the Outside region has been performed, the arm control to permit the operation may be performed. Of course, in a case where an operation to cause the distal end portion 141d transitioned to the Inside region to enter the Outside region again from a position other than the insertion port P203 has been performed, the entry of the distal end portion 141d into the Outside region is suppressed. This is similarly performed for the distal end unit 140e with the distal end portion 141e located in the Outside region. The arm control considering the entry direction into each region is performed in this way, so that assist of the operation becomes possible in accordance with the operation intended by the user. In other words, the user's operation is not impeded by the virtual boundary when the distal end unit 140 is moved from the Outside region to the Inside region, and the user's operation regarding insertion is assisted by the virtual boundary when the distal end unit 140 located on the Inside region side is inserted into the insertion port P203. Thereby, an effect of further improving the operability can be expected.

Furthermore, in the example illustrated in FIG. 16, a situation where an operation to cause the distal end portion 141 to enter the Under Trocar region from a portion where the Outside region and the Under Trocar region are in contact is performed can be assumed, as in positions P221 and P223. In such a case, entry of the distal end portion 141 into the Under Trocar region from the Outside region is suppressed.

Note that, in the examples illustrated in FIGS. 14 to 16, the Inside region corresponds to an example of the “first region”, and the Outside region corresponds to an example of “second region”.

As the first example, as an example of control regarding assist of the user operation using the virtual boundary by the system according to an embodiment of the present disclosure, the example of arm control based on the setting of the virtual boundary assuming a situation of assisting insertion of a distal end of an endoscope into a port has been described with reference to FIGS. 14 to 16.

### 2.3.6. Second Example: Operation Assist Control Example Using Virtual Boundary

As a second example, another example of control regarding assist of the user operation using the virtual boundary by the system according to an embodiment of the present disclosure will be described.

The arm control regarding assist of the user operation according to the setting of the virtual boundary according to an embodiment of the present disclosure may be set as one mode for controlling the operation of the arm device (in other words, a mode of the arm control) as illustrated in FIG. 2. In other words, as the operation mode of the arm device, the mode of the arm control according to an embodiment of the present disclosure and a mode of another arm control (for example, a mode based on a technology in related art) may be set. In this case, the mode of the arm control according to an embodiment of the present disclosure corresponds to an example of a “first mode”, and the mode of other arm control corresponds to an example of a “second mode”. As a specific example, as the operation mode of the arm device, the first mode related to assisting the user operation according to the setting of the virtual boundary based on the technology according to the present disclosure and the second mode for suppressing entry of a point of action into a predetermined region on the basis of a technology in related art (for example, a mode for preventing a contact of the distal end unit with a predetermined structure) may be set. Note that, in this case, as the second mode, the method of the arm control for suppressing the entry of the point of action into the predetermined region is not particularly limited. As a specific example, the entry of the point of action into the predetermined region may be suppressed by performing the arm control on the basis of the setting of the constraint point. Furthermore, as another example, the arm control may be performed to generate a reaction force for suppressing the entry of the point of action into the predetermined region.

Note that, in this case, an application condition of each mode can be appropriately set according to an assumable use case. As a specific example, a mode to be applied may be determined according to the distal end unit (for example, a medical instrument) held by the arm unit of the arm device. Furthermore, in a case where a plurality of configurations corresponding to the arm units is provided, a mode to be applied to each arm unit may be determined.

Furthermore, a technique of the arm control in each mode (for example, the first mode or the second mode) can be selectively applied as appropriate. For example, when suppressing the entry of the point of action (for example, the distal end unit) to a predetermined region, setting of the region and setting of the virtual boundary may be performed according to a detection result of a predetermined target such as an affected part. As a more specific example, an image analysis is applied to an image captured by an imaging unit (for example, an endoscope device) to recognize an affected part captured as a subject, and setting of a region where entry is suppressed may be performed according to the recognition result of the affected part and setting of the virtual boundary according to the setting of the region may be performed. In this case, the position of the imaging unit in the real space can be identified according to the posture of the arm unit.

As a specific example, an absolute position in the real space of the affected part captured in the image as a subject can be estimated as a relative position to the imaging unit on the basis of the identification result of the position of the imaging unit and the analysis result of the image captured by the imaging unit. Therefore, for example, a region where the affected part is located in the real space is set as a region where entry of the point of action is suppressed, and the position, posture, and shape of the virtual boundary, the position of the opening in the boundary surface of the virtual boundary, and the like can be set according to the setting of the region. Furthermore, as another example, the virtual boundary according to an embodiment of the present disclosure is set according to the setting of the insertion port for inserting an medical instrument into a body, whereby introduction of the medical instrument through the insertion port may be assisted. As a specific example, a trocar or the like is recognized to recognize the position and posture of the insertion port, and the virtual boundary may be set according to the recognition result of the position and posture of the insertion port. In this case, an opening may be set at a position corresponding to the insertion port, of the boundary surface of the virtual boundary, according to the recognition result of the position and posture of the insertion port, for example. More specifically, the shape of the boundary surface of the virtual boundary and the position of the opening in the boundary surface may be determined such that the point of action (for example, the distal end unit) inserted through the opening set in the virtual boundary is introduced into the recognized insertion port. Furthermore, control based on the detection result of a predetermined target and the detection result of a predetermined state as described above can be executed in real time, for example. In other words, the shape of the boundary surface of the virtual boundary, the position of the opening in the boundary surface, and the like may be sequentially updated according to a predetermined condition. Furthermore, as another example, the shape of the boundary surface of the virtual boundary, the position of the opening in the boundary surface, and the like may be set or updated on the basis of various triggers such as the detection of a predetermined target and the detection of a predetermined state, as described above.

Furthermore, when assisting the operation regarding the movement of the point of action toward the target position such as the insertion port, control regarding the assist may be appropriately changed. As a specific example, the assist amount regarding the movement of the point of action toward the target position may be controlled according to the positional relationship (for example, the distance or the like) between the point of action (for example, a medical instrument) and the target position (for example, the insertion port). As a more specific example, the operation of the arm unit may be controlled such that a reaction force against the movement toward the target position becomes larger as the point of action approaches the target position. Furthermore, as another example, the operation of the arm unit may be controlled such that the viscous drag coefficient regarding drive (for example, rotational movement) of each joint of the arm unit becomes higher as the point of action approaches the target position. By such control, control can be performed such that resistance regarding the movement of the point of action (in other words, resistance against the operation regarding the movement of the point of action) becomes larger as the point of action (for example, the distal end unit of the medical instrument or the like) approaches the target position. By the control, the operation of the arm unit can be made heavier or the speed regarding the movement of the arm unit (in other words, the movement of the point of action) can be restricted as the point of action approaches the target position. Therefore, the user can perform a more precise operation. Furthermore, by the above-described arm control, the user can easily recognize that the point of action is located near the target position according to the speed of the arm unit or the weight of the operation of the arm unit. Note that the arm control according to the positional relationship between the point of action and the target position may be switched on the basis of a predetermined threshold value by providing the threshold value. As a specific example, in a case where the distance between the point of action and the target position becomes equal to or smaller than the threshold value, the speed regarding the movement of the arm unit may be restricted or control may be performed such that the operation of the arm unit becomes heavier. Furthermore, the assist amount according to the movement of the point of action toward the boundary surface may be controlled according to the positional relationship (for example, the distance) between the point of action and the boundary surface of the virtual boundary on the basis of a similar idea to the above description.

Furthermore, the operation of the arm unit may be controlled such that a reaction force regarding the control of the posture of the distal end unit is generated according to the angle made by the distal end unit (for example, a medical instrument) and the boundary surface of the virtual boundary. By such control, the operation of the user can be assisted such that a long distal end unit like a lens barrel of an endoscope is more perpendicularly inserted into the insertion port, for example.

Note that the above-description is mere examples and does not necessarily limit the operation of the medical arm system according to an embodiment of the present disclosure. In other words, part of the configurations and control may be appropriately changed without departing from the idea related to the arm control, in other words, the idea related to control regarding the assist of the user operation using the virtual boundary according to an embodiment of the present disclosure.

As the second example, another example of control regarding assist of the user operation using the virtual boundary by the system according to an embodiment of the present disclosure has been described.

### 2.4 Modification

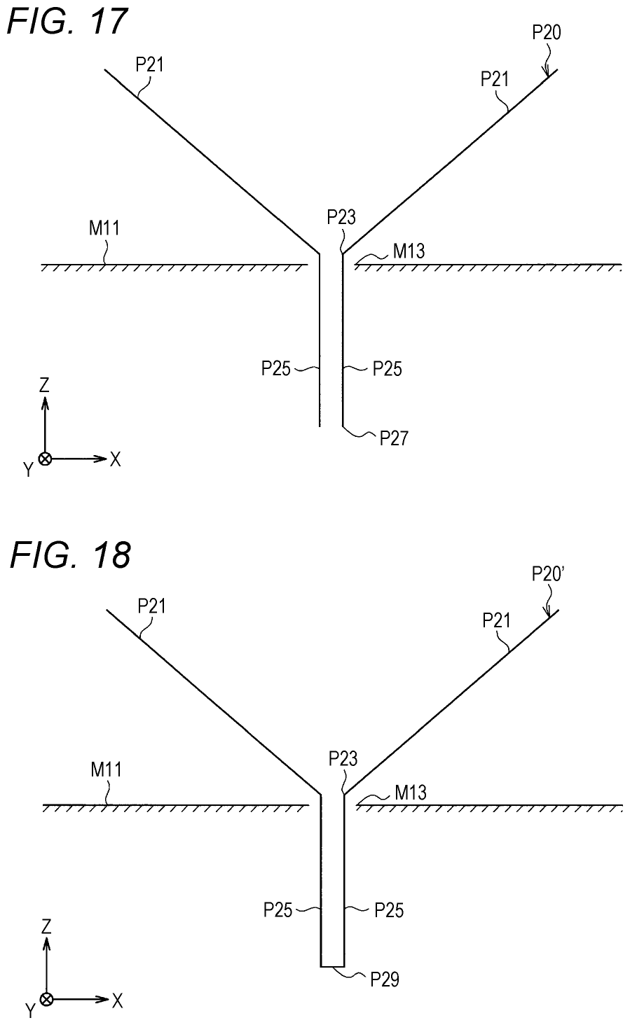

Next, modifications of the medical arm system according to an embodiment of the present disclosure will be described. In the present modifications, other examples of the virtual boundary according to an embodiment of the present disclosure will be described with reference to FIGS. 17 to 20. Note that, in the following description, the examples illustrated as FIGS. 17 to 20 are also referred to as first to fourth modifications for convenience. Furthermore, the x axis, y axis, and z axis in each of FIGS. 17 to 20 correspond to the x axis, y axis, and z axis in FIG. 5, respectively.

### 2.4.1. First Modification

First, a virtual boundary according to a first modification will be described with reference to FIG. 17. FIG. 17 is an explanatory diagram for describing an overview about a virtual boundary according to the first modification. Note that, to distinguish the virtual boundary according to the first modification illustrated in FIG. 17 from the virtual boundary according to the above-described embodiment, the virtual boundary according to the first modification is also referred to as “virtual boundary P20” for convenience.

As illustrated in FIG. 17, the virtual boundary P20 has a boundary surface P21 formed by a flat surface, a curved surface, or a combination thereof, and an opening P23 (movement target) is set in a part of the boundary surface P21. The boundary surface P21 is set to be inclined toward the opening P23. Furthermore, the virtual boundary P20 is set in the real space such that the position of the opening P23 corresponds to the position of the insertion port M13. These configurations are similar to the virtual boundary P10 described with reference to FIGS. 4 and 5.

In contrast, the virtual boundary P20 has a portion (hereinafter also referred to as “boundary surface P25”) formed such that the boundary surface P21 further extends (in other words, downward in FIG. 17) beyond the opening P23 from the portion where the opening P23 is set. Specifically, the boundary surface P25 has a tubular (for example, cylindrical) shape, and is formed to extend into the body through the insertion port M13 from the position corresponding to the opening P23. Furthermore, the boundary surface P25 is open at an end opposite to the opening P23, as indicated by reference numeral P27.

With the above configuration, the distal end portion of the distal end unit is inserted into the opening P23 as the movement toward the opening P23 is assisted along the boundary surface P21, and then the movement in the body is assisted along the boundary surface P25. In other words, the movable range of the distal end portion of the distal end unit inserted in the body through the insertion port M13 is restricted by the boundary surface P25. Thereby, the situation where the medical instrument (distal end unit) inserted in the body through the insertion port M13 comes in contact with each part in the body (for example, an organ or the like) can be prevented. Note that it is sufficient that what type of control (for example, the constraint condition, the motion purpose, or the like) is applied to the distal end unit in contact with the boundary surface P25 is appropriately determined according to the use case.

Furthermore, the shape, length, and the like of the boundary surface P25 may be appropriately changed according to the state in the body. As a specific example, assuming that a medical instrument is inserted through a nostril, the opening P23 may be set at a position corresponding to the nostril, and the boundary surface P25 may be formed along an inner side of a nasal cavity. With such a configuration, movement (insertion) of the medical instrument along the nasal cavity can be assisted while preventing occurrence of a situation where the medical device inserted into the nasal cavity through the nostril comes in contact with the inner side surface of the nasal cavity. Furthermore, change in the shapes of the nostril and nasal cavity is detected using various sensors or the like, and the position and shape of the virtual boundary P20 (in particular, the position and shape of the boundary surface P25) may be updated according to the result of the detection.

The virtual boundary according to the first modification has been described with reference to FIG. 17.

### 2.4.2. Second Modification

Next, a virtual boundary according to a second modification will be described with reference to FIG. 18. FIG. 18 is an explanatory diagram for describing an overview about a virtual boundary according to the second modification. Note that, to distinguish the virtual boundary according to the second modification illustrated in FIG. 18 from the virtual boundaries according to the above-described embodiment and other modifications, the virtual boundary according to the second modification is also referred to as “virtual boundary P20′” for convenience.

In the example illustrated in FIG. 18, reference signs P21, P23, and P25 are substantially similar to the boundary surface P21, the opening P23, and the boundary surface P25, respectively, in the example illustrated in FIG. 17. Therefore, the configuration of the virtual boundary P20′ will be described focusing on a portion different from the virtual boundary P20 illustrated in FIG. 17, and detailed description of the configuration (in other words, the boundary surface P21, the opening P23, and the boundary surface P25) substantially similar to the configuration of the virtual boundary P20 is omitted.

As can be seen by comparing FIG. 18 with FIG. 17, the virtual boundary P20′ is different from the virtual boundary P20 in FIG. 17 in being provided with an end surface P29 (in other words, being not opened) at an end opposite to the opening P23, of ends of the boundary surface P25. In other words, in the example illustrated in FIG. 18, after being inserted into the opening P23, movement of the distal end portion of the distal end unit in the body is assisted along the boundary surface P25, and when the distal end portion comes in contact with the end surface P29, further insertion is suppressed by the end surface P29. With such a configuration, insertion of the medical instrument (distal end unit) can be suppressed before the distal end of the medical instrument inserted in the body comes in contact with an organ or the like.

The virtual boundary according to the second modification has been described with reference to FIG. 18.

### 2.4.3. Third Modification

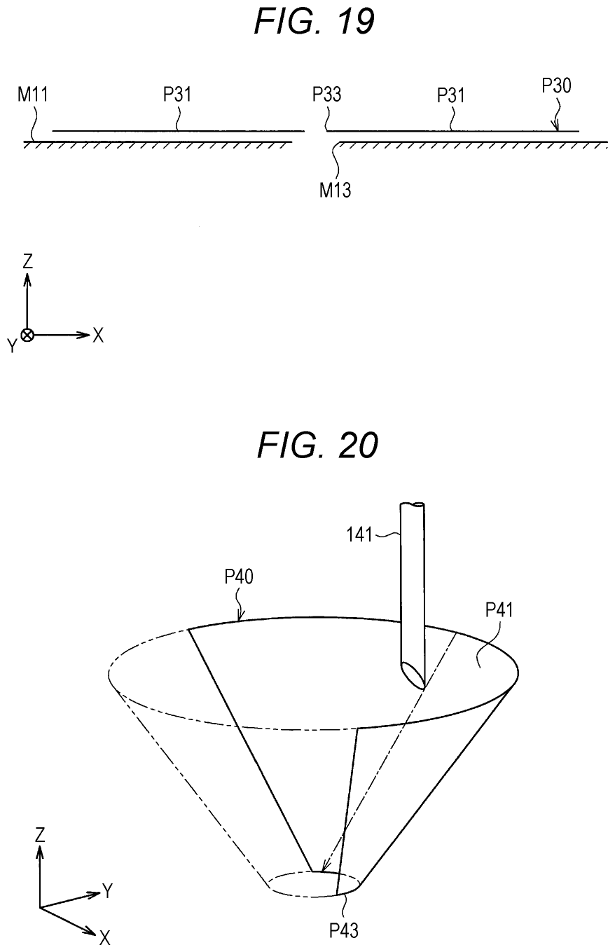

Next, a virtual boundary according to a third modification will be described with reference to FIG. 19. FIG. 19 is an explanatory diagram for describing an overview about a virtual boundary according to the third modification. Note that, to distinguish the virtual boundary according to the third modification illustrated in FIG. 19 from the virtual boundaries according to the above-described embodiment and other modifications, the virtual boundary according to the third modification is also referred to as “virtual boundary P30” for convenience.

As illustrated in FIG. 19, the virtual boundary P30 has a boundary surface P31 formed by a flat surface, a curved surface, or a combination thereof, and an opening P33 (movement target) is set in a part of the boundary surface P31. Furthermore, the virtual boundary P30 is set in the real space such that the position of the opening P33 corresponds to the position of the insertion port M13. Meanwhile, the virtual boundary P30 is different from the virtual boundaries according to the above-described embodiment and other modifications in that the boundary surface P31 is not inclined toward the opening P33.

Under such a configuration, in a case where the distal end portion of the distal end unit comes in contact with the boundary surface P31 (in other words, the distal end portion is located on the boundary surface P31), for example, the operation of the arm unit may be controlled such that the movement of the distal end unit along the boundary surface P31 toward the opening P33 (movement target) is assisted (for example, force control is performed).

Furthermore, a configuration corresponding to the boundary surface P25 may be provided to the virtual boundary P30, similarly to the examples illustrated in FIGS. 17 and 18.

The virtual boundary according to the third modification has been described with reference to FIG. 19.

### 2.4.4. Fourth Modification

Next, a virtual boundary according to a fourth modification will be described with reference to FIG. 20. FIG. 20 is an explanatory diagram for describing an overview about a virtual boundary according to the fourth modification. Note that, to distinguish the virtual boundary according to the fourth modification illustrated in FIG. 20 from the virtual boundaries according to the above-described embodiment and other modifications, the virtual boundary according to the fourth modification is also referred to as “virtual boundary P40” for convenience.

As illustrated in FIG. 20, the virtual boundary P40 has a shape in which the virtual boundary P10 illustrated in FIG. 5 is cut along a plane parallel to the z axis, and a part of the cut portion is removed. In other words, the virtual boundary P40 has a curved boundary surface P41, and one end P43 (an end in the −z direction) in a direction orthogonal to a curving direction is set as the movement target. Note that the position of the end P43 in the virtual boundary P40 corresponds to the position where the opening P13 is set in the virtual boundary P10 illustrated in FIG. 5. In other words, the boundary surface P41 is set to be inclined toward the end P43.

In other words, in a case where a cone with an apex side located downward is cut by a plane parallel to an axis of the cone and is partially removed, the virtual boundary P40 has a shape substantially equal to a remaining portion after the removal, of the side surface of the cone. That is, the cut section formed in a case where the virtual boundary P40 is cut in a plane perpendicular to the axis of the cone becomes smaller in area as the virtual boundary P40 is cut at a position closer to the end P43 (movement target).

Under such a configuration, in a case where the distal end portion of the distal end unit comes in contact with the boundary surface P41 (in other words, the distal end portion is located on the boundary surface P41), for example, the operation of the arm unit may be controlled such that the movement of the distal end unit along the boundary surface P41 toward the end P43 (movement target) is assisted. Note that the control of the operation of the arm unit regarding the assist of the movement of the distal end unit is similar to the control of the above-described embodiment and other modifications.

Furthermore, a configuration corresponding to the boundary surface P25 may be provided to the virtual boundary P40, similarly to the examples illustrated in FIGS. 17 and 18.

The virtual boundary according to the fourth modification has been described with reference to FIG. 20.

### 2.4.5. Supplement

The above-described configurations are mere examples and do not necessarily limit the configuration of the virtual boundary according to an embodiment of the present disclosure. In other words, the configuration (for example, the shape and the like) of the virtual boundary according to the present embodiment is not particularly limited as long as the virtual boundary has the boundary surface formed by a flat surface, a curved surface, or a combination thereof, and the movement target (for example, the opening) is set in part of the boundary surface. Furthermore, in the virtual boundary according to the present embodiment, it is sufficient that the movement target (for example, the opening) is set at the position corresponding to the insertion port used for inserting a medical instrument into a patient's body. A hole that penetrates the boundary surface is not necessarily provided as in the example illustrated in FIG. 18, as long as the medical instrument (distal end unit) can be inserted into the insertion port. Furthermore, the virtual boundary does not necessarily have the shape based on a perfect circular cone (or a circular truncated cone), and may be a shape based on an elliptical cone, for example. Furthermore, in a case where the virtual boundary is set to assist movement in the body, for example, movement to the target position set on an organ surface can be assisted when a surgical tool is moved to the target position. In such a case, for example, the shape of the boundary surface of the virtual boundary may be set in accordance with the shape of the organ (in other words, a shape formed along the surface of the organ). Furthermore, in such a case, it is sufficient that the movement target is set in part of the virtual boundary, and an insertable portion like the opening may not be necessarily provided in the boundary surface. In other words, the aspect of the movement target set in the virtual boundary according to an embodiment of the present disclosure is not necessarily limited to the opening.

### 3. HARDWARE CONFIGURATION

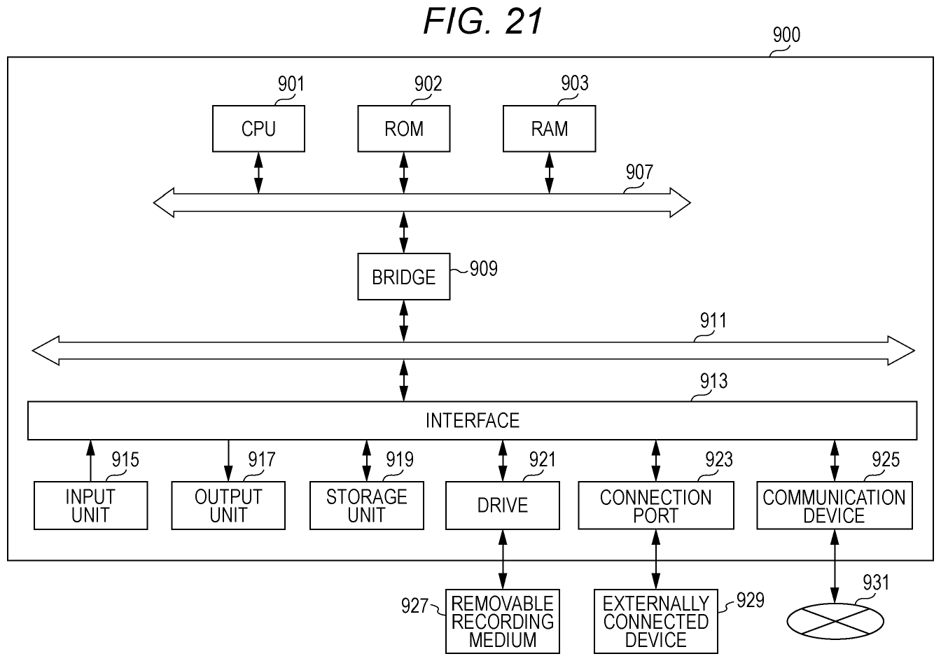

Next, an example of a hardware configuration of an information processing apparatus 900 that configures the medical arm system according to the present embodiment, like the arm device 10 and the control device 20 according to an embodiment of the present disclosure illustrated in FIG. 3, will be described. FIG. 21 is a functional block diagram illustrating a configuration example of a hardware configuration of an information processing apparatus according to an embodiment of the present disclosure.

The information processing apparatus 900 according to the present embodiment mainly includes a CPU 901, a ROM 902, and a RAM 903. Furthermore, the information processing apparatus 900 includes a host bus 907, a bridge 909, an external bus 911, an interface 913, a storage device 919, a drive 921, a connection port 923, and a communication device 925. Furthermore, the information processing apparatus 900 may also include at least one of an input device 915 or an output device 917.

The CPU 901 functions as an arithmetic processing unit and a control device, and controls the entire operation or a part thereof of the information processing apparatus 900 according to various programs recorded in the ROM 902, the RAM 903, the storage device 919, or a removable recording medium 927. The ROM 902 stores programs, arithmetic operation parameters, and the like used by the CPU 901. The RAM 903 primarily stores the programs used by the CPU 901, parameters that appropriately change in execution of the programs, and the like. The CPU 901, the ROM 902, and the RAM 903 are mutually connected by the host bus 907 configured by an internal bus such as a CPU bus. Note that, in the example illustrated in FIG. 4, the joint control unit 135 in the arm device 10 and the control unit 230 in the control device 20 can be realized by the CPU 901.

The host bus 907 is connected to the external bus 911 such as a peripheral component interconnect/interface (PCI) bus via the bridge 909. Furthermore, the input device 915, the output device 917, the storage device 919, the drive 921, the connection port 923, and the communication device 925 are connected to the external bus 911 via the interface 913.

The input device 915 is an operation unit operated by the user, such as a mouse, a keyboard, a touch panel, a button, a switch, a lever, and a pedal, for example. Furthermore, the input device 915 may be, for example, a remote control unit (so-called remote controller) using infrared rays or other radio waves or an externally connected device 929 such as a mobile phone or a PDA corresponding to an operation of the information processing apparatus 900. Moreover, the input device 915 is configured by, for example, an input control circuit for generating an input signal on the basis of information input by the user using the above-described operation unit and outputting the input signal to the CPU 901, or the like. The user of the information processing apparatus 900 can input various data and give an instruction on processing operations to the information processing apparatus 900 by operating the input device 915.

The output device 917 is configured by a device that can visually or audibly notify the user of acquired information. Such devices include display devices such as a CRT display device, a liquid crystal display device, a plasma display device, an EL display device, a lamp, and the like, sound output devices such as a speaker and a headphone, and a printer device. The output device 917 outputs, for example, results obtained by various types of processing performed by the information processing apparatus 900. Specifically, the display device displays the results of the various types of processing performed by the information processing apparatus 900 as texts or images. Meanwhile, the sound output device converts an audio signal including reproduced sound data, voice data, or the like into an analog signal and outputs the analog signal.

The storage device 919 is a device for data storage configured as an example of a storage unit of the information processing apparatus 900. The storage device 919 is configured by a magnetic storage device such as a hard disk drive (HDD), a semiconductor storage device, an optical storage device, a magneto-optical storage device, or the like, for example. The storage device 919 stores programs executed by the CPU 901, various data, and the like. Note that the storage unit 220 in the example illustrated in FIG. 4 can be realized by, for example, at least one of the ROM 902, the RAM 903, or the storage device 919 or a combination of two or more thereof.

The drive 921 is a reader/writer for a recording medium, and is built in or is externally attached to the information processing apparatus 900. The drive 921 reads out information recorded on the removable recording medium 927 such as a mounted magnetic disk, optical disk, magneto-optical disk, or semiconductor memory, and outputs the information to the RAM 903. Furthermore, the drive 921 can also write a record on the removable recording medium 927 such as the mounted magnetic disk, optical disk, magneto-optical disk, or semiconductor memory. The removable recording medium 927 is, for example, a DVD medium, an HD-DVD medium, a Blu-ray (registered trademark) medium, or the like. Furthermore, the removable recording medium 927 may be a compact flash (CF (registered trademark)), a flash memory, a secure digital (SD) memory card, or the like. Furthermore, the removable recording medium 927 may be, for example, an integrated circuit (IC) card on which a noncontact IC chip is mounted, an electronic device, or the like.

The connection port 923 is a port for being directly connected to the information processing apparatus 900. Examples of the connection port 923 include a universal serial bus (USB) port, an IEEE 1394 port, a small computer system interface (SCSI) port, and the like. Other examples of the connection port 923 include an RS-232C port, an optical audio terminal, a high-definition multimedia interface (HDMI) (registered trademark) port, and the like. By connecting the externally connected device 929 to the connection port 923, the information processing apparatus 900 directly acquires various data from the externally connected device 929 and provides various data to the externally connected device 929.

The communication device 925 is, for example, a communication interface configured by a communication device for being connected to a communication network (network) 931, and the like The communication device 925 is, for example, a communication card for a wired or wireless local area network (LAN), Bluetooth (registered trademark), a wireless USB (WUSB), or the like. Furthermore, the communication device 925 may be a router for optical communication, a router for an asymmetric digital subscriber line (ADSL), a modem for various communications, or the like. The communication device 925 can transmit and receive signals and the like, for example, to and from the Internet and other communication devices in accordance with a predetermined protocol such as TCP/IP, for example. Furthermore, the communication network 931 connected to the communication device 925 is configured by a network or the like connected by wire or wirelessly, and may be, for example, the Internet, home LAN, infrared communication, radio wave communication, satellite communication, or the like.

In the above, an example of the hardware configuration that can realize the functions of the information processing apparatus 900 according to the present embodiment of the present disclosure has been described. Each of the above-described constituent elements may be configured using general-purpose members or may be configured by hardware specialized for the function of each constituent element. Therefore, the hardware configuration to be used can be changed as appropriate according to the technical level of the time of carrying out the present embodiment. Furthermore, although not illustrated in FIG. 21, the information processing apparatus 900 may have various configurations for realizing the function according to the executable function.

Note that a computer program for realizing the functions of the information processing apparatus 900 according to the above-described present embodiment can be prepared and implemented on a personal computer or the like. Furthermore, a computer-readable recording medium in which such a computer program is stored can be provided. The recording medium is, for example, a magnetic disk, an optical disk, a magneto-optical disk, a flash memory, or the like. Furthermore, the above computer program may be delivered via, for example, a network without using a recording medium. Furthermore, the number of computers that execute the computer program is not particularly limited. For example, a plurality of computers (for example, a plurality of servers or the like) may execute the computer program in cooperation with one another.

### 4. APPLICATION

Next, application of the technology according to an embodiment of the present disclosure will be described.

As described above, the technology according to an embodiment of the present disclosure sets the virtual boundary with the opening set in part in the real space, and controls the operation of the arm unit according to the relative positional relationship between the virtual boundary and the point of action, thereby assisting the operation of the arm unit by the user. Therefore, the technology according to the present disclosure can be applied to a device and a system having a configuration corresponding to the arm unit directly or indirectly operated by the user.

For example, under the situation where the operation of the arm unit 420 of the medical arm device 400 as described with reference to FIG. 2 is controlled, the distal end unit may not necessarily be held with respect to the arm unit 420. As a specific example, a situation can be assumed, in which the distal end unit and the affected part are virtually presented to the user via a display or the like by applying a VR technology and an AR technology, and the presentation of the distal end unit is controlled in response to the operation of the arm unit by the user, whereby various procedures are simulated. In such a case, the distal end unit such as a medical instrument may not be necessarily held with respect to the arm unit operated by the user.

Furthermore, as another example, a so-called bilateral system may be configured using the medical arm system according to an embodiment of the present disclosure. The bilateral system is a system configured to control such that the posture and force state substantially match between a device operated by the user (master device) and a device performing work (slave device). As a specific example, the bilateral system performs posture control of the slave device on the basis of a user's operation on the master device, and feeds back a force detected by the slave device to the master device. More generally, whilst a master-slave device may operate in such a bilateral mode, it may also be operable in a unilateral mode, or any suitable mode; for example a collaborative mode with several masters controlling different aspects (and/or different arms) of a slave device.

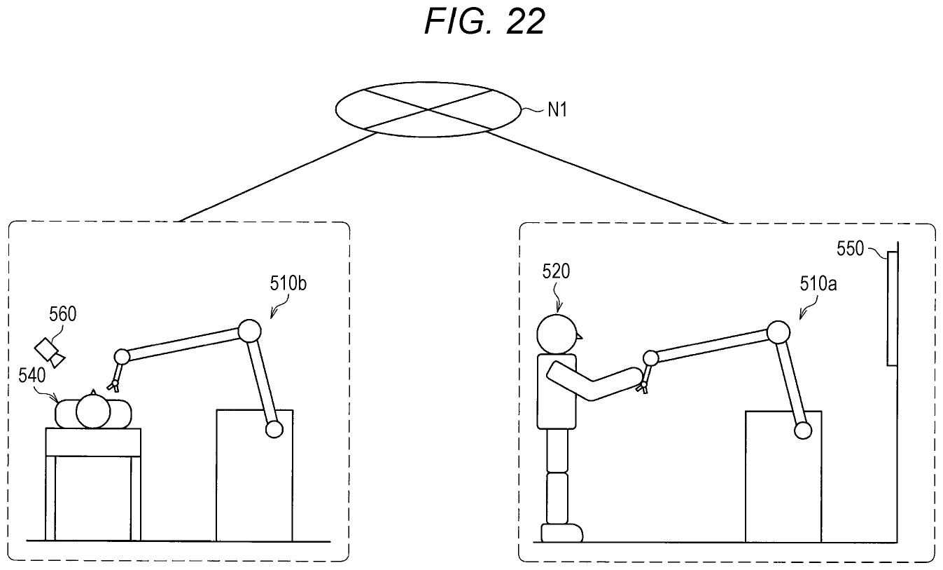

For example, FIG. 22 is an explanatory diagram for describing an application of the medical arm system according to an embodiment of the present disclosure, and illustrates an example in which a bilateral system is configured using the medical arm system. In other words, in the example illustrated in FIG. 22, an arm device 510a operating as a master device and an arm device 510b operating as a slave device are connected via a network N1. The type of the network N1 connecting the arm device 510a and the arm device 510b is not particularly limited. Under such a configuration, an image of the patient 540 located at a logically, if not necessarily physically, remote location imaged by an imaging unit 560 is presented to the practitioner 520 via the monitor 550. The remote location may, for example, be in a different hospital, the same hospital, an adjoining room (for example in a case where the medical instrument emits radiation) or the same operating room.

Furthermore, in the example illustrated in FIG. 22, control is performed such that the posture of the arm unit of the arm device 510a and the posture of the arm portion of the arm device 510b substantially coincide with each other. Specifically, when the posture of the arm unit of the arm device 510a changes in response to the operation of the practitioner 520, the posture of the arm unit is calculated. Then, the operation of the arm unit of the arm device 510b is controlled on the basis of the calculation result of the posture of the arm unit of the arm device 510a.

Under such a configuration, for example, the virtual boundary according to an embodiment of the present disclosure may be set according to the position and posture of the insertion port formed by installing a trocar or the like on the patient 540 on the side of the arm device 510b. In this case, the operation of the arm unit is controlled according to the positional relationship between the distal end unit held by the arm unit of the arm unit 510b and the virtual boundary, and the control may be fed back to the operation of the arm unit of the arm device 510a. Furthermore, a virtual boundary may be set on the side of the arm device 510a according to the situation around the arm device 510a. Note that, in a case where virtual boundaries are set for both the arm devices 510a and 510b, for example, control of the arm unit on one side (for example, control on the arm device 510b side) may be preferentially performed or the operations of the arm units may be controlled (suppressed, for example) on the basis of the states of both sides.

Furthermore, as illustrated in FIG. 22, in a system assuming remote control such as a so-called bilateral system, the distal end unit is not necessarily held to the arm unit of the arm device (that is, arm device 510a) operated by the user.

Furthermore, in the above description, the arm control according to the present embodiment has mainly been described focusing on the control of the arm unit of the medical arm device. However, the present embodiment does not limit the application destination of the arm control according to the present embodiment (in other words, an application field). As a specific example, the arm control according to an embodiment of the present disclosure can be applied to an industrial arm device. As a more specific example, a working robot provided with the arm unit is brought to enter a region where entry by a person is difficult, and the working robot can be remotely controlled, by industrially using the bilateral system as illustrated in FIG. 22. In such a case, the arm control (in other words, the control according to the setting of the virtual boundary) according to an embodiment of the present disclosure can be applied to the remote control of the arm unit of the working robot.

Furthermore, the application target of the control using the setting of the virtual boundary based on the technology according to an embodiment of the present disclosure is not necessarily limited to the arm device provided with the arm unit only. In other words, the control based on the technology according to an embodiment of the present disclosure can be applied to a device that assists the operation of the user and feeds back force sense or the like to the user according to the operation in response to the operation by the user. As a specific example, the control according to an embodiment of the present disclosure can be applied to control of a device that assists movement of each part of the user, such as a so-called robot suit. As a more specific example, it is assumed that the user wearing the robot suit performs an operation to insert a part, a tool, or the like into an insertion port formed in a desired object. At this time, a virtual boundary is set in accordance with the position and posture of the insertion port, and the drive of the robot suit is controlled in accordance with the setting of the boundary surface, whereby the operation by the user to insert a part, a tool, or the like into the insertion port can be assisted.

A control device and a medical arm system are disclosed as described herein. It will be appreciated that embodiments and options relating to the control device and/or the medical arm system and any of the wider operational environment (for example relating to medical instruments, image capture, insertion ports and the like) may be combined in any suitable manner.

Accordingly, a summary embodiment is now described, incorporating the description elsewhere herein, in which a control device (20) includes a control unit (230) adapted (for example by suitable software instruction) to control an articulated medical arm (1) configured to hold a medical instrument, where the medical instrument includes a predetermined point thereon; the control unit being adapted to control the articulated medical arm in response to a spatial relationship between the predetermined point of the medical instrument and a virtual boundary set in real space and including a target opening.

The articulated medical arm may include a multilink structure having a plurality of links connected with each other by a joint unit for example as described herein with reference at least to FIGS. 1, 2, and 3, or alternatively or in addition may include any suitable structure allowing three-axis placement of the predetermined point in at least a predetermined volume of space, such as rotation or pivot points, telescopic components, or flexible components, or any suitable combination of these.

The predetermined point (also referred to as point of action herein) refers to a predetermined point, typically on the medical instrument or on an associated extension, protrusion or consumable component of the medical instrument (such as a needle, scalpel, optical fiber, endoscope or the like), for example as described herein with reference to FIGS. 6, 7 and 8 and elsewhere. The predetermined point can act as a representative proxy for the position of some or all of the medical instrument, and is typically the point of the medical instrument (or associated part as described above) that first enters an insertion port or otherwise interacts with the patient. As described previously herein, this is done through a target opening (or ‘movement target in part’) in a virtual boundary.

The virtual boundary is a virtual surface set by the control unit, for example as described herein with reference at least to FIGS. 4, 5, and 6, having co-ordinates set with reference to a real-world position as described elsewhere herein. In some instances of the summary embodiment, the virtual surface represents a condition or trigger for actions performed by the control unit. In other instances of the summary embodiment, the virtual surface defines a virtual volume that again represents a condition or trigger for actions performed by the control unit. Consequently the control unit may control an operation of the articulated medical arm unit according to a relative positional relationship between the point of action in real space and the virtual boundary as set with reference to a point in real space.

The virtual boundary itself may be defined using any suitable representation, such as a set of polygons or voxels, or a mathematical description of a surface, such as a complete or partial surface of rotation like a cone, a convex cone (e.g. an exponential horn), or a concave cone (e.g. a bowl), or part thereof. Hence more generally the virtual boundary can comprise a slope inclined toward the target opening, the slope having a predetermined extent. The predetermined extent may for example be equivalent to a complete or partial cone wall 5 cm, 10 cm, 15 cm, 20 cm, 30 cm, or 50 cm long, or any suitable size depending on the size of the articulated medical arm system, the size of the medical instrument, and the size of the point of interaction. It will also be appreciated that the target opening may not be a circle, but may be a different aperture shape such as a slit, with the corresponding virtual boundary being for example lozenge shaped with conical walls. Similarly the target opening may be an area (i.e. more than a compact circular area such as that of an insertion port), with the virtual boundary forming a bottomless bowl with angled walls (e.g. an extrusion from a cone with a zero-dimensional apex to one describing a one dimensional line, or a two dimensional area).

The virtual boundary includes a target opening (described elsewhere herein as a movement target in part, for example with reference to FIG. 9); in other words, a null portion of the boundary, or a non-implemented portion of the boundary, or a region of space surrounded by the boundary but not part of it, acting as a hole in the boundary and a target for the medical instrument. As described elsewhere herein, the target opening is typically coincident with a point of interaction on the patient, such as an insertion port.

The virtual boundary may therefore provide a safety and/or guidance function typically centered on the point of interaction on the patient (although there is no need for the virtual boundary to be symmetrical or centered on the target opening).

Hence, in an instance of the summary embodiment the control unit is adapted to control the articulated medical arm to prevent (e.g. suppress) vertical motion of the predetermined point towards the target opening that would cause the predetermined point to pass through the virtual boundary.

Referring again to FIGS. 7, 9 and 12 and the accompanying text herein, it will be appreciated that in this case vertical motion of the predetermined point means motion on a z-axis normal or orthogonal to the target opening. Vertical motion towards the target opening therefore means movement that reduces the distance to the target opening on that z-axis. Meanwhile vertical motion towards the target opening that would cause the predetermined point to pass through the virtual boundary P11 means that the gradient of descent of the predetermined point with respect to the z-axis is greater than the gradient of the virtual boundary, so the predetermined point will cross that boundary. It will be appreciated that if the predetermined point moved parallel to the boundary, a vertical motion towards the target opening would not cause the predetermined point to pass though the boundary because in conjunction with a horizontal motion component, the overall motion vector runs parallel to the boundary.

Similarly, in an instance of the summary embodiment, the control unit is adapted to control the articulated medical arm to prevent horizontal motion of the predetermined point away from the target opening that would cause the predetermined point to pass through the virtual boundary.

Referring again to FIGS. 7, 9 and 12 and the accompanying text herein, it will be appreciated that in this case horizontal motion of the predetermined point means motion on an x-axis parallel to the target opening, and orthogonal to the z-axis mentioned above. Horizontal motion away from the target opening therefore means movement that increases the distance to the target opening on that x-axis. Meanwhile horizontal motion away from the target opening that would cause the predetermined point to pass through the virtual boundary P11 means that the gradient of ascent (if any) of the predetermined point is less than the gradient of the virtual boundary, so the predetermined point will cross that boundary. It will be appreciated that if the predetermined point moved parallel to the boundary, a horizontal motion away from the target opening would not cause the predetermined point to pass though the boundary because in conjunction with an upwards vertical motion component, the overall motion vector runs parallel to the boundary.

It will be appreciated that the medical tool including the predetermined point may optionally have any orientation whilst the predetermined point is moving; however, optionally the medical instrument of a part thereof may similarly be excluded from passing vertically or horizontally though the virtual barrier (e.g. due to rotation of the tool around the predetermined point).

In an instance of the summary embodiment, the control unit is adapted to control the articulated medical arm to prevent a predetermined motion, by generating a reaction force in the articulated medical arm equal and opposite to at least that component of an estimate of an external force being applied to the medical instrument that causes the medical instrument to exhibit the predetermined motion. The external force may be applied, for example, by a user moving the medical instrument.

Hence a vertical force contributing to a vertical motion towards the target opening that will cause the predetermined point to pass through the virtual boundary can be estimated (for example using force sensors in the arm as discussed elsewhere herein), and a reaction force to that estimated force may then be generated to counteract that estimated force and prevent the unwanted vertical motion, or vertical motion component. A feedback loop based on the position of the predetermined point with respect to the virtual boundary may be used to refine the force estimate.

For a purely vertical motion, such as that illustrated in FIG. 7, this would typically mean that all the vertical force is reacted against. Meanwhile for an angled motion with both a horizontal and a vertical motion component, such as that illustrated in FIG. 12, this would typically mean that a proportion of the applied vertical force is reacted against, so that the net vertical force, together with the applied horizontal force, produce an applied force vector parallel to the virtual boundary. This in turn causes the motion of the predetermined point to follow the virtual boundary down towards the target opening.

Similarly, a horizontal force contributing to a horizontal motion away from the target opening that will cause the predetermined point to pass through the virtual boundary can be estimated (for example using force sensors in the arm as discussed elsewhere herein), and a reaction force to that estimated force may then be generated to counteract that estimated force and prevent the unwanted horizontal motion, or horizontal motion component. A feedback loop based on the position of the predetermined point with respect to the virtual boundary may be used to refine the force estimate.

For a purely horizontal motion, this would typically mean that all the horizontal force is reacted against. Meanwhile for an angled motion with both a horizontal and a vertical motion component, such as that illustrated in FIG. 12 (but for an opposite motion to that illustrated), this would typically mean that a proportion of the applied horizontal force is reacted against, so that the net horizontal force, together with the applied vertical force, produce an applied force vector parallel to the virtual boundary. This in turn causes the motion of the predetermined point to follow the virtual boundary up and away from the target opening.

Again, in principle the orientation of the medical instrument is not considered; rather it is the vertical and horizontal components of the force applied to the medical instrument that causes the predetermined point of the medical instrument to move that are estimated. However, if the orientation of the medical instrument affects these forces, or is required to estimate these forces (for example this may be an issue if the medical instrument is flexible), then the orientation may be considered as part of the force estimation process.

In an instance of the summary embodiment, the control unit is adapted to control the articulated medical arm system to prevent a predetermined motion when the position of the predetermined point is coincident with the virtual boundary.

It will be appreciated that the above discussion of preventing vertical and/or horizontal motion through the virtual boundary means stopping the predetermined point substantially at the boundary; however, it is possible that there is a reaction delay in the medical arm device that means the reaction force should be applied before the predetermined point reaches the virtual boundary in order to stop at the virtual boundary. Similarly, the medical arm may exhibit some flexibility in response to the force being applied to the medical instrument it is holding, and so this additional force-dependent displacement (and potentially additional flexure caused by the generated reaction force) may be calculated to determine a flexure-based positional offset from the virtual boundary at which to apply the no-crossing condition.

Hence the control unit may act in advance of the predetermined point reaching the virtual boundary in order to prevent unwanted movement through the virtual boundary. Alternatively, the boundary may be considered to have a thickness or tolerance equivalent to the excess motion caused by delays in reaction force generation or arm flexure when the control unit acts in response to the predetermined point being coincident with the virtual boundary.

Meanwhile, typically horizontal and vertical motion away from the boundary (for example into the volume of space defined by the virtual boundary, or outside the range of the virtual boundary), is not prevented by a reaction force.

Hence the control unit may provide a safety function by preventing unwanted motion through the virtual boundary from an interactive region on one side of the boundary to an exclusion region on the other side of the boundary, by application of a reaction force sufficient to prevent motion at the virtual boundary, or limit motion to the gradient of the boundary.

Meanwhile, in the event that the predetermined point is found to be in the exclusion region (e.g. at a vertical position on the z-axis with respect to the target opening that is below the virtual boundary), then optionally motion towards the virtual boundary is not prevented, whilst motion further way is. For example, motion that reduces the net distance to the closest point on the boundary to the predetermined point is not prevented.

Alternatively or in addition to the safety function discussed herein, the control unit may use the virtual boundary to provide an assistance and/or guidance function for the user of the medical instrument.

Hence in an instance of the summary invention, the control unit is adapted to generate a resistive force in the articulated medical arm system that resists, but does not prevent, a movement of the predetermined point.

This may be achieved for example in a similar manner to the technique described previously herein, where components of an external force being applied to the medical instrument (or causing the medical instrument to move, for example as applied by a user) are estimated, and reaction forces are generated. However, in this instance, the generated force or forces are less than, rather than equal to, the applied force.

As a result, when such resistive force or forces are applied, it requires more effort to move, or equivalently change the position of, the predetermined point of the medical instrument. The resistive force or forces are again either vertical and/or horizontal force components, or a force vector, generated by the medial arm system under the control of the control unit. Advantageously, by requiring more force to move the predetermined point, movement can be made more accurate, with reduced jitter or wobble due to any unwanted small forces caused by the user's manual control of the medical instrument.

In principle, such accuracy of movement may become more important as the predetermined point gets closer to the target opening and hence a point of interaction with the patient. Consequently, in this instance of the summary invention, then optionally the control unit is adapted to increase the generated resistive force in the articulated medical arm as a function of the proximity of the predetermined point to the target. In other words, the control unit may be adapted to increase the generated resistive force in the articulated medical arm as the predetermined point approaches the target opening.

Again, such resistance does not prevent motion, but makes it increasingly difficult (e.g. requiring more deliberate force for a given amount of movement). Again this serves to reduce unwanted movement arising from unwanted forces such as trembling in the user's arm or hand, or small translations of force through the user's body due to breathing or shifting weight and the like. In this case, the resistance increases as the predetermined point gets closer to the target opening. This increase may be a linear or non-linear function of distance to the target opening, based either on vertical distance, horizontal distance, or a product (vector) of both.

This resistance also effectively provides a haptic feedback to the user, indicating that they are getting closer to the target opening. Other guidance may be provided in a similar manner, by imposing additional rules for the generation of resistive or reaction forces.

Hence for this instance of the summary invention, optionally the control unit is adapted to increase the generated resistive force in the articulated medical arm in response to motion outside of a predetermined range of direction. In this case, the resistive force may increase more quickly or as a step change, and optionally may increase to the point where it becomes a reaction force that prevents further motion. Hence for example, if a user is following the gradient of the virtual boundary down towards the target opening, unwanted lateral movement, or lateral movement beyond a threshold amount, or beyond a threshold amount within a threshold time period, may be additionally resisted. This acts to channel and guide the predetermined point towards the target opening. Similarly, motion back up the virtual boundary away from the target opening (e.g. reverse motion), or reverse motion beyond a threshold amount, or beyond a threshold amount within a threshold time period, may be additionally resisted. Meanwhile, an action such as moving the predetermined point towards the central volume surrounded by the virtual boundary may signify that the user is no longer intending to reach the target opening, and so such resistive forces are stopped.

It will be appreciated that other guidance rules may be implemented using such resistive and reactive forces, and/or push/pull forces. For example, it may be desirable for the medical instrument to be aligned vertically with (normal to) the target opening when it reaches the target opening. In this case, the orientation of the medical instrument, as held by the articulated medical arm, can be detected, and forces can be applied (for example as a function of distance to the target opening) to help align the medical instrument as desired. Such forces may include resisting moving out of alignment, reacting against moving out of alignment, pushing toward alignment and/or pulling toward alignment. It will also be appreciated that a guidance rule is not limited to resistance forces, but can also prevent movement in a similar manner to that described elsewhere herein in relation to the virtual boundary. Hence for example where lateral movement of the instrument is resisted, a supplementary virtual boundary may also be provided to prevent lateral movement beyond a certain deviation from a preferred path, so that even if the user ignores the guidance of the resistive force, they cannot pass the barrier. Similarly, the shape and/or contouring of the virtual boundary itself may change as a function of the position and/or movement of the predetermined point (or the medical instrument more generally) to provide guidance.

It will also be appreciated that once the predetermined point has reached the target opening, and an intervention has occurred, then safe and/or guided removal of the medical instrument is also desirable, and so the above techniques relating to controlling motion toward the target opening can be reversed as appropriate to control motion away from the target opening (e.g. in terms of guidance); meanwhile reaction forces enforcing the virtual boundary, and optionally resistive forces as a function of proximity to the target opening, may still apply as before.

Hence more generally, in an instance of the summary embodiment, the control unit is adapted to apply a generated force in the articulated medical arm in response to a guidance rule. In this instance, then as described previously the guidance rule may for example implement one or more selected from the list consisting of a path for the predetermined point toward the target opening; a path for the predetermined point away from the target opening; and an orientation of the medical instrument including the predetermined point, for example as a function of distance to the target opening.

The above guidance techniques can be applied either on or within a predetermined distance from the virtual boundary, and/or within the volume of space partially enclosed by the boundary (e.g. within the cone). Hence where the techniques are applied either on or within a predetermined distance from the virtual boundary, it may be considered that the control unit is adapted to control the articulated medical arm to modify a motion of the predetermined point when the position of the predetermined point is coincident with the virtual boundary. As noted herein, such modification may prevent, resist, push, or pull the predetermined point in a given direction, depending on the relative position of the predetermined point with respect to the boundary and/or the target opening, and on any guidance rule being implemented.

Guidance techniques may also relate to the user's interaction with the boundary itself. Hence for example in an instance of the summary embodiment, when the position of the predetermined point is coincident with the virtual boundary, the control unit is adapted to control the articulated medical arm to modify a motion of the predetermined point to maintain coincidence between the predetermined point and the virtual boundary. In other words, the control unit can apply forces to make the virtual boundary feel sticky, or magnetically attractive to, the predetermined point. This reinforces the physical feedback to the user of being on a predefined track (corresponding to the cross-section of the virtual boundary) leading toward the target opening.

Once the predetermined point reaches the target opening, the user may wish to use the associated medical instrument in a manner that is different to that used when positioning the predetermined point. Accordingly, in an instance of the summary embodiment, once the predetermined point has reached the target opening, the control unit is adapted to control the articulated medical arm to enact one selected from the list consisting of: allowing free movement of the predetermined point; and restricting further movement of the predetermined point. Which of these options (or a different option) will depend upon need (for example the nature of the medical instrument and its use). In the case of allowing free movement, this may optionally also include a progressive reduction of resistive force over a predetermined period of time, to allow a user to adapt their own control of the instrument. Optionally such free movement may however be constrained to within the perimeter of the target opening by use of reactive, forces, in a manner similar to that described previously.

Optionally, the control unit may stop control of the predetermined point altogether, or pass control of the predetermined point to a different control unit, once the predetermined point reaches the target opening, thereby limiting its control to motion of the predetermined point with reference to the virtual boundary prior to reaching the target opening.

As described previously herein, the virtual boundary is set in real space. In an instance of the summary embodiment, the virtual boundary is set in real space with reference to a target point located on a patient. Typically, the virtual boundary is set in real space so that the target opening of the virtual boundary is coincident with the target point on the patient. As described previously, the target point and the target opening can be compact (e.g. a small opening in the order of 0.5 cm to 5 cm), or extend along a path (e.g. along a planned a surgical cut), or occupy an area (e.g. for a skin graft).

Optionally, the virtual boundary can be fixed in space, for example centered on the target point. Optionally, the virtual boundary can be posed or re-posed using controls, for example via a user interface, to change a position and/or orientation of the virtual boundary as desired. Optionally, the control unit causes the virtual boundary to track the target point on the patient, for example to account for motion due to breathing, or because the patient is repositioned by medical staff, to maintain a relative positional relationship between the virtual boundary (for example the target opening) and the target point.

To achieve such tracking, in an instance of the summary embodiment the control unit is adapted to set the virtual boundary in real space responsive to image based tracking of the target point, for example as described elsewhere herein. Hence for example by determining the position and orientation of the target point (for example by recognition of a trocar or similar insertion port on the patient), the position and orientation of the virtual boundary can be set to match in real time.

In an instance of the summary embodiment the above described control device, comprising the control unit, are part of a medical arm system (such as devices 510, 400 seen in FIGS. 1 and 2) including at least one articulated medical arm configured to hold a medical instrument, as described elsewhere herein, and the control device itself. The medical arm system itself may be part of a coordinated suite of robotic devices providing assistance and/or remote operability to a user such as a surgeon.

Where the control device performs a tracking function to set the virtual boundary with respect to a target point on the patient, the medical arm system (or equivalently a separate coordinating unit, such as an overhead camera unit or other camera system supplying images or image analysis to multiple devices) includes a video camera, and an image-based tracking unit adapted to track a predetermined object, wherein for example the predetermined object is affixed to a patient (such as in the case of an insertion port or trocar).

It will be appreciated that the operation of the control device and the medical arm system as described herein constitute an example of a control method for an articulated medical arm configured to hold a medical instrument, where the medical instrument in turn comprises a predetermined point, the method comprising controlling the articulated medical arm in response to a spatial relationship between the predetermined point of the medical instrument and a virtual boundary set in real space and wherein the virtual boundary comprises a target opening.

Similarly it will be recognized that the instances of the summary embodiment described herein, and the corresponding features described elsewhere herein, similarly enact corresponding instances of a method of control.

Conversely, it will be appreciated that the above methods may be carried out on hardware suitably adapted as applicable by software instruction or by the inclusion or substitution of dedicated hardware. Thus the required adaptation to existing parts of an equivalent device may be implemented in the form of a computer program product comprising processor implementable instructions stored on a non-transitory machine-readable medium such as a floppy disk, optical disk, hard disk, solid state disk, PROM, RAM, flash memory or any combination of these or other storage media, or realised in hardware as an ASIC (application specific integrated circuit) or an FPGA (field programmable gate array) or other configurable circuit suitable to use in adapting the conventional equivalent device. Separately, such a computer program may be transmitted via data signals on a network such as an Ethernet, a wireless network, the Internet, or any combination of these or other networks.

### 5. CONCLUSION

As described above, the medical arm system according to an embodiment of the present disclosure includes the multilink structure having the plurality of links connected with each other by a joint unit, and the control unit that controls the movement of the multilink structure. The multilink structure is configured to be able to hold a medical instrument. The control unit controls the operation of the multilink structure according to the relative positional relationship between the point of action set using at least a part of the multilink structure as a reference and the virtual boundary set in the real space and having the opening in part. As a specific example, the control unit controls the operation of the multilink structure such that movement of the point of action in contact with the virtual boundary toward the opening along a surface of the virtual boundary is assisted. Furthermore, in a case of focusing on the medical arm system according to an embodiment of the present disclosure from another viewpoint, the control unit may set the virtual boundary that assists introduction of a medical instrument through the insertion port and control the operation of the multilink structure. Furthermore, in a case of focusing on the medical arm system according to an embodiment of the present disclosure from still another viewpoint, the control unit may have the first mode for assisting introduction of the medical instrument through the insertion port and the second mode for suppressing entry of the medical instrument into the region set in the real space.

With the above configurations, according to the medical arm system of an embodiment of the present disclosure, both the suppression of the operation regarding entry into a predetermined region and the improvement of the operability of the arm regarding movement to a desired position can be achieved in a favorable manner.

Although the favorable embodiments of the present disclosure have been described in detail with reference to the accompanying drawings, the technical scope of the present disclosure is not limited to such examples. It is obvious that persons having ordinary knowledge in the technical field of the present disclosure can conceive various changes and alterations within the scope of the technical idea described in the claims, and it is naturally understood that these changes and alterations belong to the technical scope of the present disclosure.

Furthermore, the effects described in the present specification are merely illustrative or exemplary and are not restrictive. That is, the technology according to the present disclosure can exhibit other effects obvious to those skilled in the art from the description of the present specification together with or in place of the above-described effects.

Note that following configurations also belong to the technical scope of the present disclosure.

(1)

A medical arm system including:

a multilink structure having a plurality of links connected with each other by a joint unit, and configured to be able to hold a medical instrument; and

a control unit configured to control an operation of the multilink structure according to a relative positional relationship between a point of action set using at least a part of the multilink structure as a reference and a virtual boundary set in a real space and having a movement target in part.

(2)

The medical arm system according to (1), in which the control unit controls the operation of the multilink structure such that movement of the point of action in contact with the virtual boundary toward the movement target along a surface of the virtual boundary is assisted.

(3)

The medical arm system according to (1) or (2), in which the virtual boundary is set such that a surface is inclined toward the movement target.

(4)

The medical arm system according to (3), in which the virtual boundary is approximately equal to a side surface of a cone or a side surface of a circular truncated cone in shape, and the movement target is set to a position corresponding to an apex of the cone or a position corresponding to at least a part of an upper surface of the circular truncated cone.

(5)

The medical arm system according to any one of (1) to (4), in which the shape of the virtual boundary is preset.

(6)

The medical arm system according to any one of (1) to (4), in which the shape of the virtual boundary is set according to a detection result of an object in the real space.

(7)

The medical arm system according to any one of (1) to (6), in which the shape of the virtual boundary is configured to be updateable.

(8)

The medical arm system according to (7), in which the shape of the virtual boundary is sequentially updated according to a predetermined condition.

(9)

The medical arm system according to (7), in which the shape of the virtual boundary is updated on the basis of a predetermined trigger.

(10)

The medical arm system according to any one of (1) to (9), in which the movement target is set according to a position of an insertion port for inserting the medical instrument into a body of a patient.

(11)

The medical arm system according to (10), in which

an opening is set as the movement target, and

the opening is set such that the medical instrument inserted in the opening is inserted into the body through the insertion port.

(12)

The medical arm system according to any one of (1) to (11), in which the virtual boundary has a surface set within a range with the movement target as a base point.

(13)

The medical arm system according to (12), in which the virtual boundary has the surface set within a region based on a range centered on the movement target.

(14)

The medical arm system according to any one of (1) to (13), in which the point of action is set to substantially coincide with a distal end of the medical instrument.

(15)

The medical arm system according to any one of (1) to (14), in which the control unit controls the operation of the multilink structure on the basis of a detection result of the point of action being located on the virtual boundary.

(16)

The medical arm system according to any one of (1) to (15), in which the control unit controls the operation of the multilink structure such that entry of the point of action into a region separated by the virtual boundary from a portion other than the movement target, of the virtual boundary, is suppressed.

(17)

The medical arm system according to (16), in which the control unit controls the operation of the multilink structure on the basis of a constraint condition regarding restriction of movement of the point of action according to a positional relationship between a constraint point serving as a reference of the control of the operation of the multilink structure, the constraint point being set in a real space according to setting of the virtual boundary, and the point of action.

(18)

The medical arm system according to (17), in which the constraint point is set on a surface of the virtual boundary.

(19)

The medical arm system according to (17) or (18), in which a position of the constraint point is updated according to a control result of the operation of the multilink structure.

(20)

The medical arm system according to (16), in which the control unit controls the operation of the multilink structure on the basis of an estimation result of an external force that acts on the virtual boundary with a contact between the virtual boundary and the point of action.

(21)

The medical arm system according to (20), in which the control unit controls the operation of the multilink structure such that a first reaction force is generated against a component that acts in a vertical direction on a surface of the virtual boundary, of the external force.

(22)

The medical arm system according to (20) or (21), in which the control unit controls the operation of the multilink structure such that a second reaction force is generated against a component that acts in a horizontal direction on the surface of the virtual boundary, of the external force.

(23)

The medical arm system according to (22), in which the control unit controls the second reaction force according to a positional relationship between the point of action in contact with the surface of the virtual boundary and the movement target.

(24)

The medical arm system according to (23), in which the control unit controls the second reaction force to be larger as a distance between the point of action and the movement target is shorter.

(25)

The medical arm system according to any one of (16) to (24), in which

the control unit

suppresses entry of the point of action from a portion other than the movement target from a first region separated by the virtual boundary toward a second region, and

permits entry of the point of action from a portion other than the movement target from the second region toward the first region.

(26)

A control device including:

a control unit configured to control an operation of a multilink structure according to a relative positional relationship between a point of action set using at least a part of the multilink structure as a reference and a virtual boundary set in a real space and having a movement target in part, the multilink structure having a plurality of links connected with each other by a joint unit, and configured to be able to hold a medical instrument.

(27)

A control method including:

by a computer,

controlling an operation of a multilink structure according to a relative positional relationship between a point of action set using at least a part of the multilink structure as a reference and a virtual boundary set in a real space and having a movement target in part, the multilink structure having a plurality of links connected with each other by a joint unit.

(28)

A program for causing a computer to execute:

controlling an operation of a multilink structure according to a relative positional relationship between a point of action set using at least a part of the multilink structure as a reference and a virtual boundary set in a real space and having a movement target in part, the multilink structure having a plurality of links connected with each other by a joint unit, and configured to be able to hold a medical instrument.

(29)

A medical arm system including:

a multilink structure having a plurality of links connected with each other by a joint unit, and configured to be able to hold a medical instrument; and

a control unit configured to set a virtual boundary for assisting movement of the medical instrument and control an operation of the multilink structure.

(30)

The medical arm system according to (29), in which the virtual boundary is a boundary for assisting introduction of the medical instrument through an insertion port.

(31)

The medical arm system according to (30), in which the control unit controls the operation of the multilink structure such that the medical instrument located on the virtual boundary moves toward the insertion port along a surface of the virtual boundary.

(32)

A control device including:

a control unit configured to set a virtual boundary for assisting insertion of a medical instrument through an insertion port, and configured to control an operation of a multilink structure having a plurality of links connected with each other by a joint unit and configured to be able to hold the medical instrument.

(33)

A control method including:

by a computer,

setting a virtual boundary for assisting insertion of a medical instrument through an insertion port, and controlling an operation of a multilink structure having a plurality of links connected with each other by a joint unit and configured to be able to hold the medical instrument.

(34)

A program for causing a computer to execute:

setting a virtual boundary for assisting insertion of a medical instrument through an insertion port, and controlling an operation of a multilink structure having a plurality of links connected with each other by a joint unit and configured to be able to hold the medical instrument.

(35)

A medical arm system including:

a multilink structure having a plurality of links connected with each other by a joint unit, and configured to be able to hold a medical instrument; and

a control unit configured to control an operation of the multilink structure, in which

the control unit has

a first mode for assisting introduction of the medical instrument through an insertion port, and

a second mode for suppressing entry of the medical instrument into a region set in a real space.

(36)

The medical arm system according to (35), including:

a plurality of the multilink structures, in which

the control unit determines, for each of the multilink structures, a mode to be applied to the control of the operation of the multilink structure.

(37)

The medical arm system according to (35), in which the control unit determines a mode to be applied to the control of the operation of the multilink structure according to the medical instrument held by the multilink structure.

(38)

The medical arm system according to any one of (35) to (37), in which the control unit sets a virtual boundary in the real space such that the entry of the medical instrument into the region set on the basis of a detection result of a position of an affected part is suppressed in the second mode.

(39)

The medical arm system according to (38), in which the control unit controls the operation of the multilink structure such that a reaction force that suppresses the entry of the medical instrument into the region is generated in the second mode.

(40)

The medical arm system according to any one of (35) to (39), in which the control unit assists the introduction of the medical instrument through the insertion port by setting a virtual boundary according to setting of the insertion port in the first mode.

(41)

The medical arm system according to (40), in which the control unit controls the operation of the multilink structure such that a movable range of the medical instrument is restricted according to a distance between the medical instrument and the insertion port.

(42)

The medical arm system according to (41), in which the control unit controls the operation of the multilink structure such that a reaction force against movement of the medical instrument toward the insertion port occurs according to the distance.

(43)

The medical arm system according to (1) or (42), in which the control unit controls the operation of the multilink structure such that a reaction force regarding control of a posture of the medical instrument occurs according to an angle made by the virtual boundary and the medical instrument.

(44)

The medical arm system according to any one of (41) to (43), in which the control unit controls the operation of the multilink structure such that a resistance regarding movement of the medical instrument occurs according to the distance between the medical instrument and the insertion port.

(45)

The medical arm system according to any one of (41) to (44), in which the control unit sets the virtual boundary on the basis of a recognition result of the insertion port based on an image analysis.

(46)

A control device including:

a control unit configured to control an operation of a multilink structure having a plurality of links connected with each other by a joint unit, and configured to be able to hold a medical instrument, in which

the control unit has

a first mode for assisting introduction of the medical instrument through an insertion port, and

a second mode for suppressing entry of the medical instrument into a region set in a real space.

(47)

A control method including:

by a computer,

controlling an operation of a multilink structure having a plurality of links connected with each other by a joint unit, and configured to be able to hold a medical instrument, and

the control method having

a first mode for assisting introduction of the medical instrument through an insertion port, and

a second mode for suppressing entry of the medical instrument into a region set in a real space.

(48)

A program for causing a computer to execute:

controlling an operation of a multilink structure having a plurality of links connected with each other by a joint unit, and configured to be able to hold a medical instrument, and

the program having

a first mode for assisting introduction of the medical instrument through an insertion port, and

a second mode for suppressing entry of the medical instrument into a region set in a real space.

(49)

A control device, including:

a control unit adapted to control an articulated medical arm configured to hold a medical instrument, where the medical instrument comprises a predetermined point thereon;

the control unit being adapted to control the articulated medical arm in response to a spatial relationship between the predetermined point of the medical instrument and a virtual boundary set in real space and including a target opening.

(50)

The control device of (49), in which

the control unit is adapted to control the articulated medical arm to prevent vertical motion of the predetermined point towards the target opening that would cause the predetermined point to pass through the virtual boundary.

(51)

The control device of (49) or (50), in which

the control unit is adapted to control the articulated medical arm to prevent horizontal motion of the predetermined point away from the target opening that would cause the predetermined point to pass through the virtual boundary.

(52)

The control device of any one of (49) to (51), in which the control unit is adapted to control the articulated medical arm to prevent a predetermined motion, by

generating a reaction force in the articulated medical arm equal and opposite to at least that component of an estimate of a force being applied to the medical instrument that causes the medical instrument to exhibit the predetermined motion.

(53)

The control device of any one of (49) to (52), in which

the control unit is adapted to control the articulated medical arm to prevent a predetermined motion when the position of the predetermined point is coincident with the virtual boundary.

(54)

The control device of any one of (49) to (53), in which

the control unit is adapted to apply a generated force in the articulated medical arm in response to a guidance rule.

(55)

The control device of (54), in which

the control unit is adapted to generate the force to assist the movement of the predetermined point in the articulated medical arm in response to the guidance rule.

(56)

The control device of any one of (49) to (55), in which

the control unit is adapted to generate a resistive force in the articulated medical arm that resists, but does not prevent, a movement of the predetermined point.

(57)

The control device of (56), in which

the control unit is adapted to increase the generated resistive force in the articulated medical arm as a function of the proximity of the predetermined point to the target opening.

(58)

The control device of (54), in which the guidance rule implements one or more selected from the list consisting of:

i. a path for the predetermined point toward the target opening;

ii. a path for the predetermined point away from the target opening; and

iii. an orientation of the medical instrument comprising the predetermined point.

(59)

The control device of any one of (54) to (58), in which

the control unit is adapted to control the articulated medical arm to modify a motion of the predetermined point when the position of the predetermined point is coincident with the virtual boundary.

(60)

The control device of any one of (49) to (59), in which once the predetermined point has reached the target opening, the control unit is adapted to control the articulated medical arm to enact one selected from the list consisting of:

i. allow free movement of the predetermined point; and

ii. restrict further movement of the predetermined point.

(61)

The control device of any one of (49) to (60)m, in which

the virtual boundary includes a slope inclined toward the target opening, the slope having a predetermined extent.

(62)

The control device of any one of (49) to (61), in which the virtual boundary is set in real space with reference to a target point located on a patient.

(63)

The control device of any one of (49) to (62), in which

the control unit is adapted to set the virtual boundary in real space responsive to image based tracking of the target point.

(64)

The control device of any one of (49) to (63), in which

when the position of the predetermined point is coincident with the virtual boundary,

the control unit is adapted to control the articulated medical arm to modify a motion of the predetermined point to maintain coincidence between the predetermined point and the virtual boundary.

(65)

A medical arm system, including:

an articulated medical arm configured to hold a medical instrument; and

a control device according to any of (49) to (63).

(66)

The medical arm system of (65), including:

a video camera; and

an image-based tracking unit adapted to track a predetermined object,

wherein the predetermined object is affixed to a patient.

(67)

A control method for an articulated medical arm configured to hold a medical instrument, where the medical instrument includes a predetermined point, the method including:

controlling the articulated medical arm in response to a spatial relationship between the predetermined point of the medical instrument and a virtual boundary set in real space and including a target opening.

(68)

A computer program including computer executable instructions adapted to cause a computer system to perform the method of (67).

(69)

A computer readable medium including the computer program of (68).

It should be understood by those skilled in the art that various modifications, combinations, sub-combinations and alterations may occur depending on design requirements and other factors insofar as they are within the scope of the appended claims or the equivalents thereof.

### REFERENCE SIGNS LIST

1 Medical arm system
10 Arm device
111 Drive control unit
120 Arm unit
130 Joint unit
131 Joint drive unit
132 Joint state detection unit
135 Joint control unit
140 distal end unit
20 Control device
220 Storage unit
230 Control unit
240 Arm state acquisition unit
250 Control condition setting unit
251 Virtual boundary update unit
253 Region entry determination unit
255 Constraint condition update unit
257 Motion purpose update unit
260 Arithmetic condition setting unit
270 Whole body coordination control unit
280 Ideal joint control unit

1. A control device, comprising:
circuitry configured to control an articulated medical arm configured to hold a medical instrument, where the medical instrument comprises a predetermined point thereon;
the circuitry being adapted to control the articulated medical arm in response to a spatial relationship between the predetermined point of the medical instrument and a virtual boundary set in real space and including a target opening.

2. The control device of claim 1, in which
the circuitry is adapted to control the articulated medical arm to prevent vertical motion of the predetermined point towards the target opening that would cause the predetermined point to pass through the virtual boundary.

3. The control device of claim 1, in which
the circuitry is adapted to control the articulated medical arm to prevent horizontal motion of the predetermined point away from the target opening that would cause the predetermined point to pass through the virtual boundary.

4. The control device of claim 1, in which
the circuitry is adapted to control the articulated medical arm to prevent a predetermined motion, by
generating a reaction force in the articulated medical arm equal and opposite to at least that component of an estimate of a force being applied to the medical instrument that causes the medical instrument to exhibit the predetermined motion.

5. The control device of claim 1, in which
the circuitry is adapted to control the articulated medical arm to prevent a predetermined motion when the position of the predetermined point is coincident with the virtual boundary.

6. The control device of claim 1, in which
the circuitry is adapted to apply a generated force in the articulated medical arm in response to a guidance rule.

7. The control device of claim 6, in which
the circuitry is adapted to generate the force to assist the movement of the predetermined point in the articulated medical arm in response to the guidance rule.

8. The control device of claim 1, in which
the circuitry is adapted to generate a resistive force in the articulated medical arm that resists, but does not prevent, a movement of the predetermined point.

9. The control device of claim 8, in which
the circuitry is adapted to increase the generated resistive force in the articulated medical arm as a function of the proximity of the predetermined point to the target opening.

10. The control device of claim 6, in which the guidance rule implements one or more selected from the list consisting of:
i. a path for the predetermined point toward the target opening;
ii. a path for the predetermined point away from the target opening; and
iii. an orientation of the medical instrument comprising the predetermined point.

11. The control device of claim 6, in which
the circuitry is adapted to control the articulated medical arm to modify a motion of the predetermined point when the position of the predetermined point is coincident with the virtual boundary.

12. The control device of claim 1, in which once the predetermined point has reached the target opening, the circuitry is adapted to control the articulated medical arm to enact one selected from the list consisting of:
i. allow free movement of the predetermined point; and
ii. restrict further movement of the predetermined point.

13. The control device of claim 1, in which
the virtual boundary comprises a slope inclined toward the target opening, the slope having a predetermined extent.

14. The control device of claim 1, in which the virtual boundary is set in real space with reference to a target point located on a patient.

15. The control device of claim 1, in which
the circuitry is adapted to set the virtual boundary in real space responsive to image based tracking of the target point.

16. The control device of claim 1, in which
under a condition the position of the predetermined point is coincident with the virtual boundary,
the circuitry is adapted to control the articulated medical arm to modify a motion of the predetermined point to maintain coincidence between the predetermined point and the virtual boundary.

17. A medical arm system, comprising:
an articulated medical arm configured to hold a medical instrument; and
a control device according to claim 1.

18. The medical arm system of claim 17, comprising:
a video camera; and
an image-based tracking unit adapted to track a predetermined object,
wherein the predetermined object is affixed to a patient.

19. A control method for an articulated medical arm configured to hold a medical instrument, where the medical instrument comprises a predetermined point, the method comprising:
controlling the articulated medical arm in response to a spatial relationship between the predetermined point of the medical instrument and a virtual boundary set in real space and including a target opening.

20. A non-transitory computer readable medium having computer executable instructions stored therein that when executed by a computer system cause the computer system to control an articulated medical arm configured to hold a medical instrument, where the medical instrument comprises a predetermined point, wherein in the control of the articulated medical arm is performed according to a spatial relationship between the predetermined point of the medical instrument and a virtual boundary set in real space and including a target opening.

21. (canceled)
